SEQUENCE LISTING (16 sequences)

SEQ ID NO: 1 - DNA, 30 bp, Artificial Sequence
  Description of Artificial Sequencedegenerate oligonucleotide used for RT-PCR
atnatnacnggnccnaayatgggnggnaar

SEQ ID NO: 2 - DNA, 26 bp, Artificial Sequence
  Description of Artificial Sequencedegenerate oligonucleotide used for RT-PCR
gcnrtytcnagcatytcnrccatraa

SEQ ID NO: 3 - DNA, 24 bp, Artificial Sequence
  Description of Artificial Sequencenested primers used for 5′ RACE
ccatgagcgagtgagtccaagaca

SEQ ID NO: 4 - DNA, 24 bp, Artificial Sequence
  Description of Artificial Sequencenested primers used for 5′ RACE
gatgacttctggatggggccatgg

SEQ ID NO: 5 - DNA, 24 bp, Artificial Sequence
  Description of Artificial Sequenceprimers for RT-PCR
ccaggtcacatggcatcagactag

SEQ ID NO: 6 - DNA, 24 bp, Artificial Sequence
  Description of Artificial Sequenceprimers for RT-PCR
ggcatagttccaggaaatgggtag

SEQ ID NO: 7 - DNA, 22 bp, Artificial Sequence
  Description of Artificial Sequencebeta-actin primer
gtgacgaggcccagagcaagag

SEQ ID NO: 8 - DNA, 22 bp, Artificial Sequence
  Description of Artificial Sequencebeta-actin primer
aggggccggactcatcgtactc

SEQ ID NO: 9 - DNA, 18 bp, Artificial Sequence
  Description of Artificial Sequencejunction
gagaagactgaggagtga

SEQ ID NO: 10 - DNA, 6 bp, Artificial Sequence
  Description of Artificial SequenceEGFP
ccatgg

SEQ ID NO: 11 - protein, 5 aa, Artificial Sequence
  Description of Artificial Sequencejunction
EKTEE

SEQ ID NO: 12 - DNA, 18 bp, Artificial Sequence
  Description of Artificial Sequencejunction
gagaagactgaggacatg

SEQ ID NO: 13 - protein, 6 aa, Artificial Sequence
  Description of Artificial Sequencejunction
EKTEDM

SEQ ID NO: 14 - DNA, 2120 bp, Mus musculus
  CDS (1541)..(1924)
tgagtccaagacatgtgagcaactgctaatgaaaagcagtaaacagccacttgggctatagcattttcaactccactctgaggtgaagattccaattacattcgagacttaagttctctcaattttctcccaacgaaagttcctgagtccagtatttacaatattacagctctagcagatcagtgcacctacaactcatctttttctgatgtatcctcttcaccagttgggggagggcctccacttccatagagtttgctgataattggctgaacaatttcttctagttccttctttttggctttaaagtcttcaatgtccgcatcctggtggctttccagccattcaatcttttcctctacagctttttccatggttctttatcttcagaagaaagtttacctcccagcttttctttatctccaatctggttcttgagagaataagcatagctttccaattcattcctggtgtcaatgcgctctttgagctttttgtcttcctcagcaaacttctcagcatcattaaccatcctttcaatttcttcaggtgtcaggcggttttggtcattggtaattgtgattttgtttttgtttcctgtacctttgtcttcagctgtcactcggagaataccattaacatctatctcaaaagtgacttcaatctggggaactccacggggagcaggaggaattccagtcagatcaaatgtacccagaagatgattgtcttttgttaggggtcgttcaccttcatagaccttgattgttacagttggctgattatcggaagccgtggagaagatctgagacttcttggtgggtaccacagtgttccttggaatcagttttgtcatgactcctcccacagtttcaataccaagtgtaaggggacaaacatcaagcagtaccagatcacctgtatcctgatcaccagagaggacaccagcctggacagcggcaccataggctacagcctcatcggggtttatgccacgggatggctccttgccattgaagaactctttcaccagttgctgaatctttggaattcgagtagatccaccaaccagaacaatttcatcaatatcagatttcctcaggctaggtccctgtccaggtcacatggcatcagactagtggtcatcttaggcttgctcctcaccatggccccatccagaagtcatctgtcttggactcactcgctcatgggccctgtctgtcctggactttctatgtagaccagaccagcttcaaactcacagagatctgtctgcctctgcctcctgagtgctgggattaaaggtatacactaccatgcctgacagtctctttcacataacacattgtgcaagtgcaaacagagttgacacatgcacattggatataaacttcaaaacccatgacctgaacatgtccatctctgagatactggttttactcctgtctttgtcaagctagttggaaaggcatttacactcttcactacccatttcctggaactatgccatctagatgccctgtatcttaatgtagaaaacatgcattttgaagttcagcatgtgaagaatacctcaagaaataaagatgcaattttgtatacttacaaactttccaggggactcacagaagaaaagaactatggattaaaagctgcagaggcctcctcacttccgtcatcgattgtcttggatgccagagacatcacaacacaaattacaaggcaaattttgcaaaatcaaaggagttcccctgagatggatagacagagagctgtgtaccatcttgccacaaggcttgtccaggccgcccgaaactctcagttggagccagacaggttacggacatacctgagtaacctcaagaagaaatatgcaggggactttcccagggctgtgggccttccagagaagactgaggagtgaccagtacacggtgtgcacaggggttgcaacttagttgctcagctttatgttttaattaaaaattatagtatattaacctcagaatggtcacctactatgaattctagatggaaaatattcattggaatagcttcaaaagggacaaatttttaaataaaagtttttgttagaaaagttaaaaaaaaaaaaaaaaaaa

SEQ ID NO: 15 - protein, 127 aa, Mus musculus
MHFEVQHVKNTSRNKDAILYTYKLSRGLTEEKNYGLKAAEASSLPSSIVLDARDITTQITRQILQNQRSSPEMDRQRAVYHLATRLVQAARNSQLEPDRLRTYLSNLKKKYAGDFPRAVGLPEKTEE

SEQ ID NO: 16 - DNA, 1005 bp, Mus musculus
  misc_feature (486): “n”=a, t, g, or c
cctgtgggtacacacaaaatataaaatacaagtatatttaaatttttttccagtccagaggattaacaacactagggcttcacactagtgctaggcaacgctcaccactgagcatgcaccagcctcagatccctagattgggcttggactaaacacagcctggaacatgatcaatttattaagagattccaggcagtaccacagcactacttgtaacaaaagtcagaaagggccatggtggtatacactttaatcccagtacctgggatcgagagacaggtggatctgagttcaaggccagcctggtctacagtgttgagttcgaggacagacaaagctaaagaccttatcttgaaaaaactaaaagcccagaaagcaattttacaaaggggaagtatgatttcaaccatgttcaaaagaattggacttaggcatcctgaaataaccctcgcctaggggagtaaacagcaaacatctctacattgntataaagataaataaatctccaagacgtggtaccaccccacagcatcacaagccccacttccctgcccaccgaagaaagacacacctgagaatccagtcttaaattttagttatctaaaataaaagatggatataattacaaacttttcacaaaaattagaccagtgtaaataacaaacatttattggtgtcacttatggtagaaaaaagttcctacaccacatgtgcatgacccaattgttaaatagaccatatccaaggtgaacacacaccctgacccacctttttctcaatttttaaaatagccaattcctcctctccctgaccccaagacatgtgagcaactgctaatgaaaagcagtaaacagccacttgggctatagcattttcaactccactctgaggtgaagattccaattacattcgagacttaagttctctcaattttctcccaacgaaagttcctgagtccagtatttacaatattacagctctagcagatcagtgcacctacaactc

(19) United States
(12) Patent Application Publication
(10) Pub. No.: US 2004/0102620 A1
(43) Pub. Date: May 27, 2004

(54) Msh4 gene splicing variant delta

(76) Inventor: Masanori Hirano, Tokyo (JP)

Correspondence Address: STERNE, KESSLER, GOLDSTEIN & FOX PLLC, 1100 NEW YORK AVENUE, N.W., WASHINGTON, DC, 20005, US

(21) Appl. No.: 10/303,689

(22) Filed: Nov. 25, 2002

(51) Int. Cl. C12Q 1/68; G01N 33/574; C07H 21/04; C12P 21/02; C12N 5/06; A61K 48/00

(52) U.S. Cl. 536/23.2; 435/6; 435/7.23; 435/69.1; 435/183; 435/320.1; 514/44

(57) ABSTRACT

An objective of the present invention is to provide an effective method for treating diseases related to apoptosis by providing a means for regulating apoptosis in cells. The present invention relates to polynucleotides comprising the nucleotide sequence of the Msh4 gene splicing variant δ that induces apoptosis (cell death). In addition, the present invention relates to a method for regulating apoptosis by using such a polynucleotide.

### FIELD OF THE INVENTION

The present invention relates to polynucleotides comprising the nucleotide sequence of the splicing variant δ of Msh4 gene that induces apoptosis (cell death) In addition, the present invention relates to a method for regulating apoptosis in cells by using the polynucleotides.

### BACKGROUND OF THE INVENTION

Two kinds of cell death are known, namely, apoptosis (cell death governed by genes) and necrosis (cell death not governed by genes). Apoptosis is also referred to as “programmed cell death” or “cellular suicide” (Tomei, et al., “Apoptosis in the APO-1 System”, Apoptosis: The Molecular Basis of Cell Death, Cold Spring Harbor Laboratory Press (1991)). Cells undergoing apoptosis are morphologically and biochemically different from cells that are undergoing necrosis. Initial definitive morphological changes associated with apoptosis as detected with an electron microscope are uniformly concentrated masses seen in the vicinity of the nuclear membrane that have condensed nuclear chromatin and a well-defined border, and also condensation of the cytoplasm. When cells undergoing apoptosis are observed under a phase contrast microscope, condensation and fragmentation of DNA, cell budding and the resulting formation of self-destructing cells can be seen.

In contrast to cell death caused by cellular disorders, apoptosis is an active process, involving self-destruction of the cell as ordered by a gene in response to a signal generated either extracellularly or intracellularly, and plays an important biological function (Kerr, J. F. R. and Searle, J., J. Pathol., 107:41 (1971)). Apoptosis, which is the physiologically and genetically regulated process of cell death, plays a central role in tissue formation and maintenance of homeostasis in multicellular organisms (Kerr, et al., Br. J. Cancer 26:239-257 (1972); Jacobson, et al., Cell 88:347-354 (1997)). Apoptosis is also a process that is indispensable for development (embryonic morphogenesis) (Michaelson, J. Biol. Rev. 62:115 (1987)). Thus, it actually plays an important role in the human body at each stage, from early embryogenesis to the unavoidable degeneration caused by aging (Syllie, A. H., Int. Rev. Cytol. 68:251 (1980)).

Massive cell death of spermatogonia is observed during spermatogenesis in mammalian seminiferous epithelium (DeRooija and Grootegoed, Curr. Opin. Cell Biol. 10: 694-701 (1998)). Cell death is also observed in neuronal development (Raff et al., Science 262: 695-700 (1993)). According to the observation by using the in situ end ligation (ISEL) technique that detects DNA fragmentation, over 50% of the cells in both the ventricular zone and the postmitotic cortical plate appear to be undergoing cell death during neurogenesis in the central nervous system (Voyvodic, Neuron 16: 693-6 (1996)). All of these programs involved in development follow a cascade of signal responses highly preserved throughout the animal world. The normal functions of the immune system, gastrointestinal system and hematopoietic system are also dependent on the normal functioning of apoptosis. The loss of normal apoptosis function is considered to be causative in diseases including cancer, viral infections, autoimmune diseases/allergies, neurodegenerative diseases and cardiovascular diseases.

The idea that tumors may occur due to inadequate apoptosis is not very old (Cope, F. O. and Wille, J., “Apoptosis”, Apoptosis: The Molecular Basis of Cell Death, Cold Spring Harbor Laboratory Press (1991)). In promotion of this idea, a new concept of cancer treatment has been unveiled in which cancer cells are destroyed through the enhancement of apoptosis in cancer cells.

At present, neoplastic diseases are mainly treated through the combined use of surgery, chemotherapy and radiotherapy. Current chemotherapy basically targets rapidly dividing tumor cells, and is ineffective against cancers that are either dormant or slowly developing. Chemotherapy also acts on non-cancer cells and causes adverse side-effects. Nearly all conventional antitumor agents demonstrate their antitumor actions by causing necrosis in tumor cells, and are unable to control the death of tumor cells genetically. On the other hand, since substances that induce apoptosis in tumor cells have the potential to be able to genetically control the death of tumor cells, research on such substances is being actively conducted. In addition, antitumor agents that induce apoptosis are expected to further enhance antitumor effects through concomitant use with conventional antitumor agents having different mechanisms of action.

As a result of the recent progress of the genomic DNA sequencing project, genes having interesting conformations are being continuously discovered (Li et al., J. Biol. Chem. 274: 11060-71 (1999); Wu and Maniatis, Cell 97: 779-90 (1999); Sugino et al., Genomics 63: 75-87 (2000); Schmucker et al., Cell 101: 671-84 (2000); Labrador et al., Nature 409: 1000(2001)). Almost all genes contain information of their amino acid sequences in only one strand of the double helix DNA. However, it was recently shown that the protein-coding information of the modifier of mdg4 gene (mod (mgd4)) of Drosophila is present in both of its complementary DNA strands (Labrador et al., Nature 409: 1000 (2001)). The mod (mgd4) gene is required during fly development and presumably assists in the establishment or maintenance of the chromatin structure. It appears that the 2.2 kb mRNA is encoded by two different strands of the DNA, in which the first four exons are encoded by one strand and the last two exons are encoded by its complementary strand, and both groups of exons are transcribed in the opposite direction. The mechanism explaining the synthesis of this 2.2 kb transcript is thought to be trans-splicing (Agabian, Cell 61: 1157-60 (1990)). In most trans-splicing cases in mammals, two precursor RNAs are transcribed from the same genomic locus (Caudevilla et al., Proc. Natl. Acad. Sci. USA 95: 12185-90 (1998); Akopian et al., FEBS Lett. 445: 177-82 (1990)). However, it has also been shown that human ACAT-1 mRNA is produced from two different chromosomes (Li et al., J. Biol. Chem. 274: 11060-71 (1999)). To accomplish this exceptional RNA processing, it is thought that the nucleus must have some structural specificity.

The cAMP responsive element modulator (CREM) and the CRE binding protein (CREB) transcription factor are typical examples of proteins having opposite functional properties that are synthesized by alternative splicing (Sassone-Corsi, Ann. Rev. Cell Dev. Biol. 11: 355-77 (1995); De Cesare et al., Trends Biochem. Sci. 24: 281-5 (1999)). The CREM gene has 11 exons; a striking feature of this gene is the presence of two basic leucine zipper (bZip) DNA-binding domains (DBDI and DBDII) that are used alternatively by differential splicing. Transcriptional activators (4 isoforms) are synthesized by alternative splicing, and consist of the glutamine-rich trans activation domain Q1 and/or Q2, a phosphorylation box (P-box) and a DBDI or DBDII domain. Repressors (3 isoforms) are also synthesized by alternative splicing, and contain neither Q1 nor Q2 domain, but have a P-box and a DBDI or DBDII. In addition, inducible cAMP early repressors (ICER; 4 isoforms) are synthesized from an alternative promoter lying within an intron, between Q2 and DBDI, and these small proteins (<120 amino acids) consist of only a DNA binding domain. CREB also has several isoforms having opposite functions (Ruppert et al., EMBO J. 11: 1503-12 (1992)) These varied proteins are involved in many complex physiological processes, including gonadal development (Sassone-Cori, Ann. Rev. Cell Dev. Biol. 11: 355-77 (1995)), circadian rhythms (Belvin et al., Neuron 22: 777-87 (1999)) and long-term memory formation (Bourtchuladze et al., Cell 79: 59-68 (1994); Maldonado et al., Proc. Natl. Acad. Sci. USA 96: 14094-9 (1999)). Thus, regulation of RNA splicing is thought to be an important factor in the generation of functional diversity in various genes.

Msh4 is a member of the MutS mismatch repair gene family and has been identified in S. cerevisiae, C. elegans, mouse and human (Ross-Macdonald and Roeder, Cell 79: 1069-80 (1994); Zalevsky et al., Genetics 153: 1271-83 (1999); Kneitz et al. Genes Dev. 14: 1085-97 (2000); Paquis-Flucklinger et al., Genomics 44: 188-94 (1997)). It has been shown that Msh4, a protein localized in the meiotic chromosomes, is required for chromosome pairing during meiosis in male and female mice (Kneitz et al., Genes Dev. 14: 1085-97 (2000)), and is suspected to form recombination nodules during pachytene in S. cerevisiae (Ross-Macdonald and Roeder, Cell 79: 1069-80 (1994)). All of MutS family members have a common functional domain that has four ATP-binding domains (Gorbalenya and Koonin, J. Mol. Biol. 213: 583-91 (1990)) and a helix-turn-helix (HTH) DNA-binding domain (Ohlendorf et al., J. Mol. Evol. 19: 109-14 (1983)).

Applicants have identified a number of splice variants for the Msh4 gene, one of which comprises an antisense RNA of the ER chaperon gene Bip/Grp78. Known examples of molecular chaperons localized in the lumen of the endoplasmic reticulum (ER) include, Bip/Grp78, GRP94, ORP150 and calreticulin. All of these play an important role in the folding of secretory proteins and membrane proteins in the ER. In addition to having a function that assists protein folding in the ER under normal conditions, ER chaperons increase quantitatively under stressful conditions and are believed to demonstrate a protective function for cells. Specifically, ER chaperons are highly expressed in cancer cells. For example, it has been reported that there is a good correlation between intracellular Bip/Grp78 levels and tumor size (Cai, J. W. et al., J. Cell Physiol. 154:229-237 (1993)). It has also been reported that the sensitivity of cancer cells to cytotoxic T cells (CTL) and tumor necrotic factor (TNF) is enhanced when the expression of Bip/Grp78 is suppressed by the antisense method (Sugawara, S. et al., Cancer Res. 53:6001-6005 (1993)), and that these cancer cells adhere poorly in mice and recede rapidly even if they become established (Jamora, C. et al., Proc. Natl. Acad. Sci. USA, 93:7690-7694 (1996)). Bip/Grp78 and ORP150, which are typical examples of ER chaperons, are expressed at comparatively high levels in the liver and pancreas (Ikeda, J. et al., Biochem. Biophys. Res. Commun., 230:94-99 (1997))

Even in yeast and mammalian cells, the accumulation of misfolded polypeptides in the ER lumen (ER stress) causes an unfolded-protein response (UPR). Namely, the transcription levels of genes coding for ER chaperons and folding catalysts localized in the nucleus are upregulated (Kaufmann, R., Gene Dev., 13:1211-1233 (1999)). Genes that have been regulated by this UPR contain regulatory factors preserved in their promoters (known as UPRE in yeast and ERSE in higher eucaryotic cells).

Similar to many polypeptides transported by secretory pathways, amyloid precursor protein (APP) interacts with BiP in the ER (Yang, Y. et al., J. Biol. Chem. 273:25552-25555 (1998)). The binding between BiP and APP is transitory, and is limited to a form that exists in the immature core-glycosylated ER of APP. Excessive expression of BiP decreases maturation of APP to the modified form observed in Golgi bodies, and suppresses the formation of Aβ40 and Aβ42 peptides from wild APP and its Swedish variant (which has been indicated to correlate with Alzheimer's disease) (Yang, Y. et al., J. Biol. Chem. 273:25552-25555 (1998)).

### SUMMARY OF THE INVENTION

An objective of the present invention is to provide a means for regulating apoptosis in cells, and to provide an effective method for treating diseases related to apoptosis.

The inventors of the present invention identified mouse Msh4 gene, and clarified its specific genomic structure in the present invention. As a result, seven variants were found to exist for the Msh4 gene. Among these, the cDNA of variant δ has an extremely interesting structure comprising an antisense RNA of the ER chaperon gene, Bip/Grp78 (Munro and Pelham, Cell 46:291-300 (1986)) and a short ORF of 127 amino acid residues identical to the C-terminal amino acid sequence of other Msh4 variants. The first three exons and the remaining exons of this variant are located on different chromosomes, and it has been clarified that the gene demonstrates unexpected functions due to the extremely strange type of splicing of the Msh4 gene.

The mouse ER chaperon Bip/Grp78 gene, which is one of the five hsp70 family members, has already been mapped to 21-23.5 cM of chromosome 2 (Hunt et al., Genomics 16: 193-8 (1993)). Because the 1.1 kb sequence of Msh4 gene splicing variant δ cDNA is perfectly complementary to the Bip/Grp78 mRNA sequence determined already (Haas and Meo, Proc. Natl. Acad. Sci. USA 85: 2250-4 (1988)) and the first two introns of variant δ are complementary to the last two introns of Bip/Grp78, the spliceosome must recognize the reverse splice donor and acceptor site (ct-ac) of precursor RNA and exactly splice out these introns (reverse splicing). The next splicing should be carried out in trans between the donor site of Bip/Grp78 antisense RNA precursor and the acceptor site of Msh4 RNA precursor, and then mature chimeric RNA will be completed by following ordinary splicing of four introns.

The Msh4 splicing variant δ mRNA is the first example in which antisense precursor RNA is processed by reverse splicing to form longer complementary RNA to messenger RNA including protein coding region. Further, its maturation is completed by another unusual interchromosomal trans-splicing. Human MSH4 gene also has a deductive open reading frame of C-terminal 127 amino acids, and may be trans-spliced in a manner similar to that of the mouse MSH4 gene.

In the transient co-transfection experiments of the Msh4 gene splicing variant δ ORF-EGFP fusion and CAGS-nlacZ plasmids, the inventors have found that constitutive expression of antisense RNA of the Bip/Grp78 gene causes cell death. On the contrary, deletion and inversion of Bip/Grp78 antisense RNA region of variant δ cDNA have little or no effect of killing cells. These observations suggest that cell death is a result of the formation of a double strand RNA between the 5′ half region of variant δ mRNA and 3′ half region of Bip/Grp78 mRNA that has been shown to be ubiquitously expressed (Ringwald et al., Nucleic Acids Res. 29: 98-101 (2001); Mouse genome informatics accession ID: MGI:1204866).

The molecular mechanism of the initial event of this cell-killing phenomenon may be explained by the RNA degradation process known as RNA interference (RNAi) or inhibition of translation. RNAi is a gene silencing phenomenon triggerd by double stranded (ds) RNA. The observations of the present invention suggest the possibility that the silencing of the ER chaperon Bip/Grp78 by RNAi triggers cell death.

The present invention involves the recognition of the fact that the Msh4 gene splicing variant δ mRNA identified herein ultimately causes cell death. Namely, the present invention relates to the following polynucleotides and their uses.

(1) An isolated polynucleotide that forms a double-stranded moiety with an mRNA encoding ER chaperon Bip/Grp78, said polynucleotide selected from the group consisting of:

(i) the nucleotide sequence set forth in SEQ ID NO: 14;

(ii) the nucleotide sequence of (i), wherein one or more residues have been deleted, inserted, substituted and/or added to a 5′ half of said sequence, the 5′ half comprising nucleotide 1-1098 in the nucleotide sequence set forth in SEQ ID NO:14;

(iii) a nucleotide sequence complementary the nucleotide sequence of (i) that hybridizes thereto under stringent conditions, wherein said stringent hybridization conditions comprise: prehybridizing for at least 30 minutes at 68° C., adding a labeled probe and hybridizing for at least 1 hour at 68° C., followed by washing three times for 20 minutes at room temperature in 2×SSC, 0.01% SDS, then three times for 20 minutes at 37° C. in 1×SSC, 0.1% SDS, and finally twice for 20 minutes at 50° C. in 1×SSC, 0.1% SDS;

(iv) a nucleotide sequence having 90% or more homology with a 5′ half of the nucleotide sequence of (i) and 50% or more homology with a 3′ half of the nucleotide sequence of (i), wherein the line between the 5′ and 3′ halves of the sequence arises at nucleotide 1098 of SEQ ID NO:14; and

(v) a polynucleotide having a nucleotide sequence comprising at least 25 consecutive nucleotides selected from nucleotides 1-1098 in the nucleotide sequence set forth in SEQ ID NO: 14, wherein said fragment is perfectly complementary to a nucleotide sequence corresponding to the mRNA encoding Bip/Grp78.

(2) A DNA encoding the splicing variant δ mRNA of Msh4 gene.

(3) An expression vector comprising the DNA of (2).

(4) The expression vector of (3), wherein the DNA of (2) is linked downstream of a tumor-specific promoter and/or a tumor-specific transcription regulatory sequence.

(5) A pharmaceutical composition comprising an active agent selected from the group consisting of the polynucleotide of (1), the DNA of (2), the expression vector of (3), and the expression vector of (4).

(6) The pharmaceutical composition of (5), wherein said active agent is present in an amount effective to induce or promote apoptosis in cancer cells.

(7) A method for regulating apoptosis in a target cell, wherein the method comprises the step of introducing into the target cell a construct comprising a polynucleotide selected from the group consisting of:

(i) the nucleotide sequence set forth in SEQ ID NO: 14;

(ii) the nucleotide sequence of (i), wherein one or more residues have been deleted, inserted, substituted and/or added to a 5′ half of said sequence, the 5′ half comprising nucleotide 1-1098 in the nucleotide sequence set forth in SEQ ID NO:14;

(iii) a nucleotide sequence complementary the nucleotide sequence of (i) that hybridizes thereto under stringent conditions, wherein said stringent hybridization conditions comprise: prehybridizing for at least 30 minutes at 68° C., adding a labeled probe and hybridizing for at least 1 hour at 68° C., followed by washing three times for 20 minutes at room temperature in 2×SSC, 0.01% SDS, then three times for 20 minutes at 37° C. in 1×SSC, 0.1% SDS, and finally twice for 20 minutes at 50° C. in 1×SSC, 0.1% SDS;

(iv) a nucleotide sequence having 95% or more homology with a 5′ half of the nucleotide sequence of (i) and 50% or more homology with a 3′ half of the nucleotide sequence of (i), wherein the line between the 5′ and 3′ halves of the sequence arises at residue 1098 of SEQ ID NO:14; and

(v) a polynucleotide having a nucleotide sequence comprising at least 25 consecutive nucleotides selected from nucleotides 1-1098 in the nucleotide sequence set forth in SEQ ID NO: 14, wherein said fragment is perfectly complementary to a nucleotide sequence corresponding to the mRNA encoding Bip/Grp78.

(8) A method for detecting the activity of a test compound to promote or suppress apoptosis, wherein the method comprises the following steps of:

(i) constructing a cell that expresses the splicing variant δ mRNA of Msh4 gene,

(ii) contacting the cell constructed in (i) with a test compound,

(iii) measuring the level of apoptosis in the cells and comparing it to a control, and,

(iv) detecting an apoptosis-promoting activity of the compound when the apoptosis level measured in step (iii) is increased as compared to the control, and detecting an apoptosis-suppressing activity when the apoptosis level measured in step (iii) is decreased a's compared to the control.

(9) A method of screening for a compound having an activity to promote or suppress apoptosis, comprising:

(i) detecting the apoptosis activity of a test compound by the method of (8), and

(ii) selecting the compound that promotes or suppresses apoptosis.

(10) A nucleotide probe comprising at least 25 consecutive nucleotides from the nucleotide sequence set forth in SEQ ID NO: 14, wherein said probe hybridizes under stringent conditions to the splicing variant δ mRNA of Msh4 gene, said stringent hybridization conditions comprising: prehybridizing for at least 30 minutes at 68° C., adding a labeled probe and hybridizing for at least 1 hour at 68° C., followed by washing three times for 20 minutes at room temperature in 2×SSC, 0.01% SDS, then three times for 20 minutes at 37° C. in 1×SSC, 0.1% SDS, and finally twice for 20 minutes at 50° C. in 1×SSC, 0.1% SDS.

(11) A kit for detecting apoptosis in cells by detecting the presence of the splicing variant δ mRNA of Msh4 gene comprising the nucleotide probe of (10).

(12) A method for detecting apoptosis in cells comprising the step of detecting the presence of the splicing variant δ mRNA of Msh4 gene by the nucleotide probe of (10).

(13) A polynucleotide that (a) comprises at least 25 consecutive nucleotides from the nucleotide sequence set forth in SEQ ID NO: 16, and (b) regulates the transcription of the splicing variant δ of Msh4 gene.

### BRIEF DESCRIPTION OF THE DRAWINGS

FIG. 1 schematically represents the structure of Msh4 variant δ cDNA. A box filled with diagonal lines indicates the open reading frame (ORF) deduced from the cDNA sequence. White boxes show non-coding sequences. Variant δcontains antisense RNA of ER chaperon Bip/Grp78 gene (GenBank, D78645) on its 5′-side and a small ORF on its 3′-side. Helix-turn-helix (HTH) and a functional domain containing the Nuclear Localization Signal (NLS)-like sequence are also shown.

FIG. 2 schematically shows the genomic structure of mouse Msh4 variant δ gene. The top horizontal lines represent mouse genomic DNA. Short horizontal lines indicate the corresponding λFixII genomic DNA clones and vertical lines show XbaI sites. The exon-intron structure of variant δ is schematically represented on the bottom. In variant δ, a part of the exon(s) is located at a chromosome different from the Msh4 locus. Chromosomal locations of the mouse Msh4 and Bip/Grp78 genes have already been determined (GenBank, AC087184; Hunt et al., Genomics 16: 193-8 (1993)). The chromosomal location of the 5′ UTR of the Msh4α (first exon) is currently unknown. In humans, the chromosomal locations of the MSH4 and BIP/GRP78 genes have already been determined (Paquis-Flucklinger et al., Genomics 44: 188-94 (1997); Hendershot et al., Genomics 20: 281-4 (1994)).

FIG. 3 shows the exon-intron junction sequences of the mouse Msh4 variant δ gene. Bold letters indicate the consensus splice donor and acceptor sequences. The exon sizes (bp) are indicated within parentheses.

FIG. 4 shows the expression of the Msh4 variant δ mRNA as detected using Northern blot hybridization and RT-PCR. (A) Northern blot analysis of mouse Msh4 gene expression using variant r cDNA as a probe. (B) RT-PCR analysis of Msh4 variant expression using a variant δ specific primer pair. To examine whether amplified products are correct, Southern blot hybridizations were carried out using internal probes. Amplified products were mapped by restriction endonucleases.

FIG. 5 shows NIH3T3 cells transiently transfected with an expression vector containing a fusion gene of Msh4 variant δ ORF and EGFP. The arrowhead in panel 2 indicates the region emitting fluorescence. Panel 3 shows expression of β-galactosidase containing a nuclear localization signal. Panels 4 and 5 show expression of the variant δ ORF-EGFP fusion protein with deletion and inversion mutations in the Bip/Grp78 antisense RNA region, respectively. Scale bars=5 μm.

FIG. 6 shows the result of the constitutive expression of the variant δ mRNA. (A) Co-transfection of variant δ ORF-EGFP fusion plasmid containing inversion mutations of the Bip/Grp78 antisense RNA region (C53-2CE1) and CAGS-nlacZ plasmid. Over 600 of β-galactosidase expressing cells were observed. (B) Co-transfection of variant δ ORF-EGFP fusion plasmid containing deletion mutations of the Bip/Grp78 antisense RNA region (C53-1CE1) and CAGS-nlacZ plasmid. 100 to 200 of β-galactosidase expressing cells were observed. (C) Co-transfection of the variant δ ORF-EGFP fusion plasmid containing wild type of the Bip/Grp78 antisense RNA region (C53CE1) and CAGS-nlacZ plasmid. Only several cells expressing β-galactosidase were observed. Scale bars=5/m.

FIG. 7 shows the nucleotide sequence of the promoter region of variant δ. An arrow is attached to the sequence that is extremely similar to the sequence of the Heat Shock Element (HSE) of human Hsp70 gene.

### DETAILED DESCRIPTION OF THE INVENTION

Polynucleotides of the present invention comprise either the nucleotide sequence set forth in SEQ ID NO: 14, degenerates thereof, or a sequence complementary thereto. Nucleic acid base sequences that can form a double-stranded structure through hydrogen bonding of matching base pairs are designated as “complementary”. The polynucleotides of the present invention have a sequence capable of forming double-strand RNA with ER chaperon Bip/Grp78 mRNA and are not necessarily limited to the sequence described in SEQ ID NO: 14. For example, a polynucleotide that contains a sequence in which one or more nucleotides have been deleted, inserted, substituted and/or added in the nucleotide sequence described in SEQ ID NO: 14 is included in the polynucleotide of the present invention. This type of polynucleotides can be produced by preparing synthetic DNA (or RNA) using a chemical technique or by inserting a site-specific mutation and so forth.

In addition, a polynucleotide that hybridizes with the nucleotide sequence set forth in SEQ ID NO: 14 under stringent conditions is also included in the polynucleotides of the present invention. This type of polynucleotide can be isolated from mammalian (including humans) tissues or cells in which the polynucleotide of the present invention is presumed to be expressed, by hybridization techniques using as a probe the nucleotide sequence of Bip/Grp78 mRNA, a sequence complementary to the sequence indicated in SEQ ID NO: 14, or a portion of these sequences. Examples of tissues that can be used include the testis and embryonic head.

A person with ordinary skill in the art is able to suitably select stringent conditions for hybridization. For example, stringent conditions for hybridization are disclosed in Sambrook, J. et al., Molecular Cloning 2nd ed., 9.47-9.58, Cold Spring Harbor Lab. Press (1989). More specifically, after a prehybridization for at least 30 minutes at 68° C. using a commercially available kit such as Rapid-hyb Buffer (Amersham Life Science), a labeled probe is added followed by hybridization by warming for at least 1 hour at 68° C. Subsequently, washing is done three times for 20 minutes at room temperature in 2×SSC, 0.01% SDS, then three times for 20 minutes at 37° C. in 1×SSC, 0.1% SDS, and finally twice for 20 minutes at 50° C. in 1×SSC, 0.1% SDS. A person with ordinary skill in the art is able to achieve a similar degree of stringency by taking into account several conditions, including temperature and salt concentration.

Moreover, the polynucleotides of the present invention also include those that contain a sequence having high sequence identity to the sequence of SEQ ID NO: 14. In the present invention, a high sequence identity refers to at least an identity of 50% or more, more preferably, 55%, 65%, 70%, 75%, 80%, 85%, 90%, 95%, 96%, 97%, 98%, 99%, or more sequence identity to the sequence of SEQ ID NO: 14. Identity of a nucleotide sequence can be determined according to the BLAST algorithm of Karlin and Altschul (Proc. Natl. Acad. Sci. USA, 90: 5873-5877 (1993)). BLASTN and other programs have been developed based on this algorithm (Altschul, et al., J. Mol. Biol., 215: 403-410 (1990)). When analyzing a nucleotide sequence by BLASTN based on BLAST, for example, the parameters are score=100 and wordlength=12. In the case of using the BLAST or Gapped BLAST program, the default parameters of each program are used. Specific techniques for such analytical methods are already known (see the website of the “National Center for Biotechnology Information”).

The polynucleotides of the present invention can be prepared by known methods. They can be synthesized chemically as previously mentioned, or may be isolated from tissues or cells by the hybridization technology, or can be amplified using PCR. For example, whole mRNA is first prepared from testis by a known method such as guanidine ultracentrifugation (Chirgwin, J. M. et al., Biochemistry 18: 5294-5299 (1979)) or AGPC (Chomczynski, P. and Sacchi, N., Anal. Biochem. 162: 156-159 (1987)), followed by purification of mRNA from whole RNA using a commercially available kit such as the mRNA Purification Kit (Pharmacia). Alternatively, mRNA may be prepared directly using, for example, the QuickPrep mRNA Purification Kit (Pharmacia).

Next, cDNA is synthesized using reverse transcriptase from the resulting mRNA. Alternatively the cDNA can also be synthesized using a kit such as the AMV Reverse Transcriptase First-strand cDNA Synthesis Kit (Seikagaku Co., Ltd.). In addition, a primer for synthesizing full-length cDNA can be designed based on the sequence set forth in SEQ ID NO: 14. By then carrying out PCR using this designed primer and a cDNA library as the template, cDNA comprising the nucleotide sequence shown in SEQ ID NO: 14 can be amplified. Moreover, the target cDNA can be isolated by cloning the amplification product.

The target cDNA fragment is prepared from the resulting cDNA and inserted into suitable vector DNA. A polynucleotide having a function equivalent to the splicing variant δ mRNA of Msh4 gene of the present invention can also be obtained by inserting the vector into E. coli or other suitable host cell followed by transcription.

In the present invention, a “polynucleotide having a function equivalent to the splicing variant δ mRNA of Msh4 gene” refers to being able to induce apoptosis by forming a double strand with the ER chaperon Bip/Grp78 mRNA. When producing a polynucleotide by transcription in host cells using cDNA, DNA can be altered by commercially available kits and known methods. Examples of alterations include alteration by digestion using restriction enzymes, insertion of a synthetic oligonucleotide or DNA fragment, addition of a linker, and insertion of an initiation codon (ATG) and stop codon (TAA, TGA or TAG). In addition, the present invention provides an Msh4 gene splicing variant δ mRNA or a DNA encoding a polynucleotide having an equivalent function. Namely, a polynucleotide selected from the group consisting of the following (ii) through (iv) that has a function that is equivalent to the Msh4 gene splicing variant δ mRNA is a preferable polynucleotide in the present invention:

(ii) a polynucleotide comprising a nucleotide sequence obtained by deleting, inserting, substituting and/or adding one or more nucleotides in the nucleotide sequence set forth in SEQ ID NO: 14;

(iii) a polynucleotide that hybridizes under stringent conditions with a polynucleotide comprising the nucleotide sequence set forth in SEQ ID NO: 14; and,

(iv) a polynucleotide comprising a nucleotide sequence having 50% or more identity to the nucleotide sequence of SEQ ID NO: 14.

A polynucleotide derived from human is an example of a polynucleotide that has a function equivalent to the Msh4 gene splicing variant δ mRNA of the present invention. A polynucleotide derived from human can be expected to be safer and more effective when administering to humans. A human-derived polynucleotide having a function equivalent to the Msh4 gene splicing variant δ mRNA can be acquired from a human cDNA library based on the nucleotide sequence information of SEQ ID NO: 14 (mouse) according to the above methods. More specifically, the target polynucleotide can be acquired by hybridization screening or cloning based on PCR.

In addition, the present invention provides an expression vector comprising a polynucleotide of the present invention. When the vector is used to produce a polynucleotide of the present invention as mentioned above, E. coli (JM109, DH5α, HB101, XL1Blue, etc.) and so forth can be used as a host for retaining and/or producing polynucleotide. In order to produce a polynucleotide in E. coli, there are no particular restrictions on the vector, provided that it has an “ori” and retains a marker for selecting the transformed E. coli. A drug resistance gene and so forth that enables screening using a drug may be used for the marker. Typical examples of selective drugs that are used include but are not limited to ampicillin, tetracycline, kanamycin and chloramphenicol. More specifically, vectors such as M13, vectors, pUC vectors, pBR322, pBluescript or pCR-Script can be given as vectors that can be used in the present invention. In addition, when using for the purpose of sub-cloning or cutting out cDNA, vectors such as pGEM-T, pDIRECT and pT7 can also be used in addition to those mentioned above.

Moreover, when producing the Msh4 gene splicing variant δ mRNA or a polynucleotide having a function equivalent to it by transcribing DNA in E. coli, it is necessary to have a suitable promoter, such as the lacZ promoter (Ward, et al., Nature 341: 544-546 (1989); FASEB J. 6: 2422-2427 (1992)), araB promoter (Better, et al., Science 240: 1041-1043 (1988)) or T7 promoter. In addition to the above vectors, examples of such vectors include GEX-5X-1 (Pharmacia), QIAexpress system (Qiagen), pEGFP and pET. When using a vector containing such a promoter, E. coli that expresses T7 RNA polymerase is preferably used for the host. More specifically, BL21 and so forth can be used. The vector can be introduced into the host cells by the calcium chloride method, electroporation and so forth.

In addition to E. coli, other examples of hosts include mammalian cells, insect cells, plant cells, yeasts and other bacterial cells such as Bacillus subtilis. Vectors such as those indicated below can be used in the present invention:

Expression vectors derived from mammals (pcDNA3 (Invitrogen), pEGF-BOS (Nucleic Acids Res., 18(17): 5322 (1990), pEF, pCDM8, etc.)

Expression vectors derived from animal viruses (pHSV, pMV, pAdexLcw, etc.)

Expression vectors derived from retroviruses (pZIPneo, etc.)

Vectors derived from insect cells (Bac-to-BAC baculovirus expression system (Gibco BRL), pBacPAK8, etc.)

Expression vectors derived from plants (pMH1, pMH2, etc.)

Expression vectors derived from yeast (Pichia Expression Kit (Invitrogen) pNV11, SP-Q01, etc.), and

Expression vectors derived from Bacillus subtilis (pPL608, pKTH50, etc.)

Examples of bacterial expression systems are described in Chang (Nature 275: 615 (1978)), Goeddel (Nature 281: 544 (1979); Nucleic Acids Res., 8: 4057 (1980)), Published European Patent Application (EP-A) No. 36776, U.S. Pat. No. 4,551,433, deBoer (Proc. Natl. Acad. Sci. USA 80: 21-25 (1983)) and Siebenlist (Cell 20: 269 (1980)).

The systems described in Hinnen (Proc. Natl. Acad. Sci. USA 75: 1929 (1978)), Ito (J. Bacteriol. 153: 163 (1983)), Kurtz (Mol. Cell Biol. 6: 142 (1986)), Kunze (J. Basic Microbiol. 25: 141 (1985)), Glesson (J. Gen. Microbiol. 132: 3459 (1986)), Roggenkamp (Mol. Gen. Genet. 202: 302 (1986)), Das (J. Bacteriol. 158: 1165 (1984)), DeLouvencourt (J. Bacteriol. 154: 737 (1983)), Van der Berg (Bio/Technology 8: 135 (1990)), Cregg (Mol. Cell Biol. 5: 3376 (1985)), U.S. Pat. No. 4,837,148, U.S. Pat. No. 4,929,555, Beach and Nurse (Nature 300: 706 (1981)), Davidow (Curr. Genet. 10: 380 (1985)), Gaillardin (Curr. Genet. 10: 49 (1985)), Balance (Biochem. Biophys. Res. Commun. 112: 284-289 (1983)), Tilburn (Gene 26: 205-221 (1983)), Yelton (Proc. Natl. Acad. Sci. USA 81: 1470-14.74 (1984)), Kelley and Hynes (EMBO J. 4: 475-479 (1985)), EP-A No. 244234 and Published PCT Application No. WO91/00357 are included as yeast expression systems.

Examples of insect cell expression systems are described in U.S. Pat. No. 4,745,051, Friesen (“The Regulation of Baculovirus Gene Expression”: The Molecular Biology of Baculoviruses (W. Doerfler, ed.)), EP-A Nos. 127839 and 155476, Vlak (J. Gen. Virol. 69: 765-776 (1988)), Miller (Ann. Rev. Microbiol. 42: 117 (1988)), Carbonell (Gene 73: 409 (1988)), Maeda (Nature 315: 592-594 (1985)), Lebacq-Verheyden (Mol. Cell Biol. 8: 3129 (1988)) Smith (Proc. Natl. Acad. Sci. USA 82: 8404 (1985)), Miyajima (Gene 58: 273 (1987)) and Martin (DNA 7: 99 (1988)). A large number of baculovirus strains, variants and corresponding permissible insect host cells are described in Luckow (Bio/Technology 6: 47-55 (1988)) and Miller (Genetic Engineering, Setlow, J. K. et al., ed., Vol. 8, Plenum Publishing (1986) pp. 277-279).

Examples of mammalian expression systems are described in Dijkema (EMBO J. 4: 761 (1985)), Gorman (Proc. Natl. Acad. Sci USA 79: 6777 (1982)), Boshart (Cell 41: 521 (1985)) and U.S. Pat. No. 4,399,216. Exogenous genes can be expressed in mammals according to methods described in Ham and Wallace (Meth. Enzymology 58: 44 (1979)), Barnes and Sato (Anal. Biochem. 102: 255 (1980)), U.S. Pat. Nos. 4,767,704, 4,657,866, 4927762 and 4560655, and Published PCT Application Nos. WO90/103430 and WO87/00195.

When a vector is used for the purpose of expressing in animal cells such as CHO cells, COS cells or NIH3T3 cells, a promoter is necessary. Examples of suitable promoters that can be used include but are not limited to the SV40 promoter (Mulligan, et al., Nature 277: 108 (1979)), the MMLV- the LTR promoter, the EF1α promoter (Mizushima, et al., Nucleic Acids Res. 18: 5322 (1990)) and CMV promoter. Moreover, it is preferable that the vector has a marker gene for selecting transformed cells. For example, genes such as drug resistance genes that enable screening by drugs (e.g., neomycin and G418) can be used as marker genes. Examples of vectors having such characteristics include pMAM, pDR2, pBK-RSV, pBK-CMV, pOPRSV and pOP13.

In addition, in animal cells, the expression of exogenous genes can be stabilized, and the number of gene copies in cells can be increased. For example, a method is known in which a vector having the DHFR gene that compensates for a deficiency in nucleic acid synthesis pathway is inserted into CHO cells having the same deficiency, which is then followed by enhancing gene expression by methotrexate (MTX). A known example of a preferable vector for this method is pCHOI. On the other hand, when the objective is the transient expression of a gene, a method can be used in which COS cells having a gene that expresses SV40 T antigen on their chromosomes are transformed with a vector having the replication origin of SV40 (e.g., pcD). A replication origin derived from polyoma virus, adenovirus or bovine papilloma virus (BPV) and so forth can be used. Moreover, in order to increase the number of gene copies in the host cell system, the expression vector can also contain aminoglycoside transferase (APH) gene, thymidine kinase (TK) gene, E. coli xanthine guanine phosphoribosyl transferase (Ecogpt) gene or dihydrofolate reductase (dhfr) gene and so forth as a selection marker.

A polynucleotide of the present invention can also be introduced into a living body and expressed in the living body. Expression in the living body can be used as a means for gene therapy and so forth using the Msh4 gene splicing variant δ mRNA or a polynucleotide having a function equivalent to it. There are various known methods for introducing nucleic acids into the body (Friedman, T., Science 244: 1275-1281 (1989)).

A vector for inserting a DNA encoding a polynucleotide into an animal body can be constructed by commonly used methods (Sambrook, et al. edit., Molecular Cloning: A Laboratory Manual, 5.61-5.63, Cold Spring Harbor Laboratory (1989)), using known vectors derived from a plasmid, phage, cosmid, virus, etc.

For example, administration to mammals, including humans, can be carried out using a virus vector such as a retrovirus vector (e.g., pZIPneo), adenovirus vector (e.g., pAdexlcw), vaccinia virus vector, poxvirus vector, adeno-associated virus vector, herpes virus vector, Semliki forest virus vector, astrovirus vector, HVJ vector, coronavirus vector, orthomyxovirus vector, papovavirus vector, paramyxovirus vector, parvovirus vector, picornavirus vector, togavirus vector and alphavirus vector (see K. Adolph, “Viral Genome Methods”, CRC Press, Florida (1996); Jolly, Cancer Gene Therapy 1: 51-64 (1994); Kimura, Human Gene Therapy 5: 845-852 (1994); Connelly, Human Gene Therapy 6: 185-193 (1995); Kaplitt, Nature Genetics 6: 148-153 (1994); Miller, Human Gene Therapy 15-14 (1990); Friedman, Science 244: 1275-1281 (1989); Eglitis and Anderson, Biotechniques 6: 608-614 (1988); Tolstoshev and Anderson, Curr. Opin. Biotech. 1: 55-61 (1990); Sharp, Lancet 337: 1277-1278 (1991); Cornetta, et al., Nucl. Acids Res. Mol. Biol. 36: 311-322 (1987); Anderson, Science 226: 401-409 (1984); Moen, Blood Cells 17: 407-416 (1991); Miller et al., Biotech. 7: 980-990 (1989); Le Gal Le Salle, et al., Science 259: 988-990 (1993); and Johnson, Chest 107: 77S-83S (1995)).

Retrovirus vectors are well known in the field of gene therapy, and any retrovirus gene therapy vector, including type B, type C or type D retroviruses, xenotropic viruses (e.g., NZB-X1, NZB-X2 and NZB9-1 (O'Neill, J. Vir. 53: 160 (1985)), polytropic retroviruses (e.g., MCF and MCF-MLV (Kelley, J. Vir. 45: 291 (1985)), spumavirus and lentivirus, can be used (see RNA Tumor Viruses, 2nd Edition, Cold Spring Harbor Laboratory (1985)). Retrovirus gene therapy vectors may be constructed by taking different parts from different retroviruses. For example, the long terminal repeats (LTRs) of a retrovirus can be taken from the mouse sarcoma virus, the tRNA bonding site from the Rous sarcoma virus, the packaging signal from the mouse leukemia virus and the starting point of second strand synthesis can be taken from the avian leukemia virus.

Next, the above retrovirus is introduced into a suitable packaging cell line to produce transfection-competent retroviral particles (U.S. patent application Ser. No. 07/800,921). The retrovirus vector can be incorporated site-specifically into host cell DNA by incorporating chimeric integrase in the retrovirus particles (see U.S. patent application Ser. No. 08/445,466). Here, the recombinant virus vector is preferably a replication-deficient recombinant virus. A suitable packaging cell line to be used with the retrovirus vectors described above that is known in field of this invention may be prepared according to known methods (see U.S. patent application Ser. No. 08/240,030 and WO92/05266), and may be used to produce a cell line that itself produces recombinant vector particles. The packaging cell line is preferably produced from a human parent cell (e.g., HT1080 cells) or mink parent cell line.

Examples of retroviruses that are suitable for constructing the retrovirus gene therapy vector include avian leukemia virus, bovine leukemia virus, mouse leukemia virus, mink cell focus-forming virus, mouse sarcoma virus, retinal endothelial virus and Rous sarcoma virus. Particularly preferable examples of mouse leukemia virus include 4070A and 1504A (Hartley and Rowe, J. Virol. 19: 19-25 (1976)), Abelson (ATCC No. VR-999), Friend (ATCC No. VR-245), Graffi, Gross (ATCC No. VR-590), Kirsten, Harvey sarcoma virus and Rauscher Retrovirus. Examples of known retrovirus vectors that can be used in the context of the present invention are described in GB2200651, EP415731, EP345242, WO89/02468, WO89/05349, WO89/09271, WO90/02806, WO90/07936, WO94/03622, WO93/25698, WO93/25234, WO93/11230, WO93/10218, WO91/02805, U.S. Pat. No. 5,219,740, Vile (Cancer Res. 53: 3860-3864 (1992); Cancer Res. 53: 962-967 (1992)), Ram (Cancer Res. 53: 83-88 (1993)), Takamiya (J. Neurosci. Res. 33: 493-503 (1992)), Baba (J. Neurosurg. 79: 729-735 (1993)), Mann (Cell 33: 153 (1983)), Cane (Proc. Natl. Acad. Sci. USA 81: 6349 (1984)) and Miller (Human Gene Therapy I (1990)).

Human adenovirus gene therapy vectors are also known in the art (see Berkner, Biotechniques 6: 616 (1988); Rosenfeld, Science 252: 431 (1991); WO93/07283; WO93/06223 and WO93/07282). In addition to those described above, known adenovirus gene therapy vectors that can be used in the present invention also include those described in WO94/12649, WO93/03769, WO93/19191, WO94/28938, WO95/11984, WO95/00655, WO95/27071, WO95/34671, WO96/05320, WO94/08026, WO94/11506, WO93/06223, WO94/24299, WO95/14102, WO95/24297, WO95/02697, WO94/28152, WO95/09241, WO95/25807, WO95/05835, WO94/18922 and WO95/09654. In addition, the above vectors can also be administered by coupling a DNA to the inactivated adenovirus as described in Curiel, et al., Hum. Gene Ther. 3: 147-154 (1992)).

The gene delivery vehicle of the present invention includes an adeno-associated virus (AAV) vector. A preferable example of such a vector suitable for use in the present invention is the AAV-2 basal vector disclosed in Srivastava (WO93/09239). Examples of other AAV vectors that can be used include, but are not limited to, pWP-19 and pWN-1 (Nahreini, Gene 124: 257-262 (1993)). Moreover, SSV9AFABKneo, which contains AFP enhancer and albumin promoter, is also known (Su, Human Gene Therapy 7: 463-470 (1996)), and can be preferentially expressed in the liver. Other AAV gene therapy vectors are described in U.S. Pat. Nos. 5,354,678, 5,173,414, 5139941 and 5252479.

Herpes vector may be included as a vector for gene therapy in the context of the present invention. An example is the herpes simplex virus vector disclosed in U.S. Pat. No. 5,288,641 and EP176170 (Roizman), which contains a sequence coding for thymidine kinase polypeptide. Other examples of herpes simplex virus vectors include HFEM/ICP6-LacZ disclosed in Published PCT Application No. WO95/04139 (Wistar Institute), pHSVlac disclosed in Geller (Science 241: 1667-1669 (1988)), WO90/09441 and WO92/07945, HSVUs3::pgC-lacZ described in Fink (Human Gene Therapy 3: 11-19 (1992)), HSV7134, 2RH105 and GAL4 described in EP453242 (Breakefield), and those deposited with ATCC as ATCC No. VR-977 and ATCC No. VR-260.

Moreover, alphavirus gene therapy vectors can also be used. A preferred example of alphavirus vectors is sindbis virus vector. Examples of alphaviruses include, but are not limited to, togavirus, Semliki forest virus vector (ATCC VR-67; ATCC VR-1247) , Middleberg virus (ATCC VR-370), Ross river virus (ATCC VR-373; ATCC VR-1246), Venezuelan equine encephalomyelitis virus (ATCC VR-923; ATCC VR-1250; ATCC VR-1249; ATCC VR-532) and those described in U.S. Pat. Nos. 5,091,309 and 5,217,879 and WO92/10570.

Examples of other virus vectors suitable for gene therapy using a polynucleotide having a function equivalent to the Msh4 gene splicing variant δ mRNA of the present invention include, but are not limited to, polio virus (ATCC VR-58; vectors described in Evans, Nature 339: 385 (1989) and Sabin, J. Biol. Standardization 1: 115 (1973)), rhinovirus (ATCC VR-1110; vectors described in Arnold, J. Cell Biochem. L401 (1990)), pox viruses such as canary pox virus and vaccinia virus (ATCC VR-111; ATCC VR-2010; vectors described in Fisher-Hoch, Proc. Natl. Acad. Sci. USA 86: 317 (1989), Flexner, Ann. NY Acad. Sci. 569: 86 (1989), Flexner, Vaccine 8: 17 (1990), U.S. Pat. No. 4,603,112, U.S. Pat. No. 4,769,330 and WO89/01973)), SV40 virus (ATCC VR-305; vectors described in Mulligan, Nature 277: 108 (1979) and Madzak, J. Gen. Vir. 73: 1533 (1992)), influenza virus (ATCC VR-79; U.S. Pat. No. 5,166,057, Enami, Proc. Natl. Acad. Sci. USA 87: 3802-3805 (1990), Enami and Palese, J. Virol. 65: 2711-2713 (1991) and Luytjes, Cell 59: 110 (1989)), recombinant influenza virus produced using reverse direction gene technology (McMicheal, NE J. Med. 309: 13 (1983); Yap, Nature 273: 238 (1978); Nature 277: 108 (1979)), human immunodeficiency virus (such as vectors described in EP-A No. 386882 and Buchschacher, J. Vir. 66: 2731 (1992)), measles virus (ATCC VR-67; ATCC VR-1247; vectors described in EP-A No. 440219), aura virus (such as ATCC VR-368) Bebaru virus (such as ATCC VR-600; ATCC VR-1240), Cabassou virus (such as ATCC VR-922), Chikungunya virus (such as ATCC VR-64; ATCC VR-1241), Fort Morgan virus (such as ATCC VR-924), Getah virus (such as ATCC VR-369; ATCC VR-1243), Kyzylagach virus (such as ATCC VR-1244), Mayaro virus (such as ATCC VR-66) Mucambo virus (such as ATCC VR-580, ATCC VR-1244), Ndumu virus (such as ATCC VR-371), Pizuna virus (such as ATCC VR-372; ATCC VR-1245), Tonate virus (such as ATCC VR-925), Triniti virus (such as ATCC VR-469), Una virus (such as ATCC VR-374), Whataroa virus (such as ATCC VR-926), Y-62-33 virus (such as ATCC VR-375) O'Nyong virus, Eastern encephalitis virus (such as ATCC VR-65; ATCC VR-1242), Western encephalitis virus (such as ATCC VR-70; ATCC VR-1251; ATCC VR-622; ATCC VR-1252), and corona virus ATCC VR-740; vectors described in Hamre, Proc. Soc. Exp. Biol. Med. 121: 190 (1966)).

In addition, a non-viral approach can be used to introduce a gene into the living body. For example, the polynucleotide or DNA of the present invention can be administered by any of the following:

The HVJ liposome method,

The liposome method,

The cationic liposome method (U.S. Pat. No. 5,422,120; U.S. Pat. No. 4,762,915; WO95/13796; WO94/23697; WO91/14445; EP-A No. 524968; Stryer, Biochemistry 236-40 (1975); W. H. Freeman, San Francisco, Szoka, Biochem. Biophys. Acta 600: 1 (1980); Bayer, Biochem. Biophys. Acta 550: 464 (1979); Rivnay, Meth. Enzymol. 149: 119 (1987); Wang, Proc. Natl. Acad. Sci. USA 84: 7851 (1987); Plant. Anal. Biochem. 176: 420 (1989)),

directly administering a nucleic acid of the present invention by injection and so forth (naked DNA) (WO90/11092; WO93/03709; U.S. Pat. No. 5,580,859; Roussel, Nature 325: 549 (1987)),

The calcium phosphate method (Chen C. and Okayama H., Mol. Cell Biol. 7: 2745-2752 (1987)),

The DEAE-dextran method (Lopata M. A. et al., Nucleic Acids Res. 12: 5707-5717 (1984); Sussman D. J. and Milman G., Mol. Cell Biol. 4: 1642-1643 (1985)),

Electroporation (Chu G. et al., Nucleic Acids Res. 15: 1311-1326 (1987)),

introducing by a gene gun after coating onto colloidal gold particles or other beads (carrier) (U.S. Pat. No. 5,149,655),

Lipofection (Felgner et al., Proc. Natl. Acad. Sci. USA 84: 7413 (1987); Ono et al., Neurosci. Lett. 117: 259 (1990); Brigham et al., Am. J. Med. Sci. 298: 278 (1989); Staubinger et al., Meth. Enz. 101: 512 (1983); Derijard B., Cell 7: 1025-1037 (1994); Lamb B. T. et al., Nature Genetics 5: 22-30 (1993); Rabindran S. K. et al., Science 259: 230-234 (1993)),

Asialoorosomucoid-polylysine conjugate (Wu et al., J. Biol. Chem. 263: 14621 (1998); Wu et al., J. Biol. Chem. 264: 16985 (1989); Wu and Wu, J. Biol. Chem. 262: 4429-4432 (1987); Hucked, Biochem. Pharmacol. 40: 253-263 (1990); Plank, Bioconjugate Chem. 3: 533-539 (1992)),

Polycationic condensed DNA (Curiel, Hum. Gene Ther. 3: 147-154 (1992)),

Ligand-coupled DNA (Wu, J. Biol. Chem. 264: 16985-16987 (1989), and

Microinjection during surgery (Wolff et al., Science 247: 1465 (1990)).

To express the Msh4 gene splicing variant δ mRNA of the present invention or a polynucleotide having a function equivalent to it in the body, DNA may be expressed using any promoter, and expression can be controlled by a suitable mammalian regulating element. For example, DNA of the present invention may be expressed using an enhancer that is known to regulate expression either tissue-specifically or cell-specifically as necessary.

When using the Msh4 gene splicing variant δ mRNA of the present invention, or a polynucleotide having a function equivalent to it, in the treatment of a tumor in particular, DNA coding for the polynucleotide may be linked so as to be controlled by a tumor-specific promoter or tumor-specific transcription regulating sequence. Such tumor-specific promoters and tumor-specific transcription regulating sequences selectively induce genes under their control in target tumor cells or at a level higher than in normal cells. For example, the following can be given as examples of tumor-specific transcription regulatory sequences:

secretory leukoprotease inhibitor (SLPI) (targets various cancers; Garver R. I., Gene Therapy 1: 46 (1994)),

tyrosinase (targets melanoma; Vile R. et al., Gene Therapy 1: 307 (1994); WO94/16557),

mouse serglycin (hematopoietic cell-specific TR element; U.S. Pat. No. 5,340,739),

ap2 adipose enhancer (targets adipose cells; Graves R. A. et al., J. Cell. Biochem. 49: 219 (1992)),

α1-antitrypsin transthyretin (targets liver cancer; Grayson D. R. et al., Science 239: 786 (1988)),

IL-10 (mRNA level) (targets polymorphic glioblastoma; Nitta T. et al., Brain Res. 649: 122 (1994)),

c-erbB-2 (targets pancreatic cancer, breast cancer, stomach cancer, ovarian cancer and non-small cell lung cancer; Harris J. D. et al., Gene Ther. 1: 170 (1994)),

αB-crystallin/heat shock protein (HSP) 27 (targets brain tumors; Aoyama A. et al., Int. J. Cancer 55: 760 (1993)),

basic fibroblast growth factor (bPGF) (targets glioma and meningioma; Shibata F. et al., Growth Fact. 4: 277 (1991)),

epidermal growth factor receptor (EGFR) (targets squamous cell carcinoma, glioma and breast cancer; Ishii S. et al., Proc. Natl. Acad. Sci. USA 82: 4920 (1985)),

mucinoid glycoproteins (DF3, MUC1) (targets breast cancer; Abe M. and Kufe D., Proc. Natl. Acad. Sci. USA 90: 282 (1993)),

mts1 (targets metastatic tumors; Tulchinsky E. et al., Proc. Natl. Acad. Sci. USA 89: 9146 (1992)),

Stromelysin 3 (targets breast cancer; Okada T. et al., Proc. Natl. Acad. Sci. USA 92: 2730 (1995)),

NSE (targets small cell lung cancer; Forss-Petter S. et al., Neuron 5: 187 (1990)),

Somatostatin receptor (NPI) (targets small cell lung cancer; Bombardieri E. et al., Eur. J. Cancer 31A: 184 (1995); Koh T. et al., Int. J. Cancer 60: 843 (1995)),

Aromatic L-amino acid decarboxylase (AADC) (targets at neuroectodermal tumors; Thai A. L. V. et al., Mol. Brain Res. 17: 227 (1993)),

c-erbB-3 and c-erbB-2 (targets breast cancer; Quin C. M. et al., Histopathology 25: 247 (1994); Gasparihi P. et al., Eur. J. Cancer 30A: 16 (1994)),

c-erbB-4 (targets breast cancer and stomach cancer; Rajkumar T. et al., Breast Cancer Res. Treat 29: 3 (1994); Pear C. J. et al., Year Immunol. 7: 182 (1993)),

thyroglobulin (targets thyroid adenocarcinoma; Mariotti S. et al., J. Clin. Endocrinol. Meth. 80: 468 (1995)),

α-fetoprotein (targets liver cancer; Zuibel I. et al., J. Cell Physiol. 162: 36 (1995)),

villin (targets stomach cancer; Osborn M. et al., Virchows Arch. A. Pathol. Anat. Histopathol. 413: 303 (1988)),

reg (pancreatic calculus protein) (targets colon cancer, rectal tumor, pancreatic cancer and kidney cancer; Watanabe T. et al., J. Biol. Chem. 265: 7432 (1990)),

parathyroid hormone-related peptide (PTHrP) (targets at liver cancer, cecal tumor, schwannoma, kidney cancer, pancreatic cancer and adrenocortical cancers; Campos R. V. et al., Mol. Rnfovtinol. 6: 1642 (1991)), and

albumin (targets liver cancer; Huber B. E., Proc. Natl. Acad. Sci. USA 88: 8099 (1991)).

In addition, other examples of tumor-specific promoters are described in Gotoh et al., The Journal of Urology 160: 220-229 (1998), Pang S. et al., Cancer Research 157: 495-499 (1997), Lin C. S. et al., J. Biol. Chem. 268(4): 2793-2801 (1993) and Lin Ching-Shwun et al., DNA and Cell Biology 16(I): 9-16 (1997)).

Moreover, the present invention relates to a pharmaceutical composition comprising the Msh4 gene splicing variant δ mRNA, a polynucleotide having a function equivalent to it, a DNA coding for the polynucleotide, a fragment of it, or an expression vector containing the DNA. Such pharmaceutical compositions can be used as gene therapy agents. When using the pharmaceutical composition of the present invention as a gene therapy agent, since it is necessary to induce temporary apoptosis locally, it is preferable that the pharmaceutical composition be administered in such a way that the mRNA is transiently expressed. Namely, the polynucleotide of the present invention or a vector that expresses the polynucleotide is preferably applied directly by injection and so forth to the desired apoptosis site. However, it may also be made to ultimately arrive at the desired affected tissue (e.g., a cancerous tissue) following intravascular administration.

Moreover, a polynucleotide of the present invention or a construct that comprises the polynucleotide can be used to regulate apoptosis in cells. A “construct that comprises the polynucleotide of the present invention” in the present specification includes an expression vector that comprises the Msh4 gene splicing variant δ mRNA, or a DNA coding for the mRNA, or a polynucleotide having antisense effects on the mRNA. By inserting the construct of the present invention in the form of an expression vector that comprises the Msh4 gene splicing variant δ mRNA, or DNA coding for the mRNA, into cells, apoptosis can be induced in the cells.

In addition to a polynucleotide having a function equivalent to Msh4 gene splicing variant δ mRNA, a fragment thereof can also be used in the method for inducing apoptosis of the present invention as well as in a pharmaceutical composition for that purpose. Among the fragments that can be used in the present invention, a fragment that contains a nucleotide sequence complementary to the Bip/Grp78 gene is particularly important. In the nucleotide sequence set forth in SEQ ID NO: 14, the nucleotide sequence from nucleotides 1-1098 is composed of the antisense sequence of Bip/Grp78. As indicated in the Examples, apoptosis inducing activity of the Msh4 gene splicing variant δ is either decreased or lost due to deletion or inversion in this region. Thus, a fragment of a polynucleotide that comprises a nucleotide sequence comprising this region may be useful in a method that regulates apoptosis and in a pharmaceutical composition of the present invention. Furthermore, when using a fragment of the sequence of the Msh4 gene splicing variant δ in a method for regulating apoptosis of the present invention or in a pharmaceutical composition of the present invention, there are no restrictions on the length of the fragment, provided the fragment has apoptosis inducing activity. A fragment is thought to be able to cause apoptosis as long as it has at least 25 bp of the Msh4 gene splicing variant δ.

In particular, a fragment comprising a nucleotide sequence selected from the nucleotide sequence from nucleotides 1-1098 is preferable as a fragment used in a method for regulating apoptosis or in a pharmaceutical composition of the present invention. In the present invention, a fragment that usually comprises 25 bp or more, preferably 30 bp or more, and more preferably 40 bp or more is used. More specifically, a sequence fragment that typically has 100 bp or more, preferably 200 bp or more, and more preferably 500 bp or more can be used.

Since it has the ability to induce apoptosis, a polynucleotide or a fragment thereof having a function that is equivalent to the Msh4 gene splicing variant δ mRNA of the present invention can be applied to various diseases. Examples of applicable diseases include, but are not limited to, HTLV-I-related diseases, such as cancer, AIDS, AIDS-related conditions (ARC), Adult T-cell leukemia (ATL), hairy cell leukemia, myelopathy (HAM/TSP), respiratory disorders (HAB/HABA), arthropathy (HAAP) and uveitis (HAU), autoimmune diseases, such as systemic lupus erythematosus (SLE), collagen diseases, such as chronic rheumatoid arthritis (RA), ulcerative colitis, Sjögren's syndrome, primary biliary cirrhosis, idiopathic thrombocytopenic purpura (ITP), autoimmune hemolytic anemia, myasthenia gravis, Hashimoto's disease and insulin-dependent (type I) diabetes.

Moreover, a pharmaceutical composition of the present invention that induces apoptosis can be used for diseases that accompany decreased platelet levels, in which by inhibiting the proliferation of blast cells, the compound would be able to promote the proliferation of mature cells. Thus, the pharmaceutical composition of the present invention can be used for diseases such as osteomyelodysplasia, cyclic thrombocytopenia, aplastic anemia, idiopathic thrombocytopenia and disseminated intravascular coagulation.

Moreover, other examples of applicable diseases include various types of hepatitis, including type A, type B, type C and type F, myocarditis, adult respiratory distress syndrome (ARDS), infections, cirrhosis, prostatomegaly, hysteromyoma, bronchial asthma, arterial sclerosis, various congenital malformation, nephritis, senile cataract, chronic fatigue syndrome and muscular dystrophy. Moreover, a method has also been proposed for preventing opacification, a major complication following lens transplantations, by removing contaminating epithelial cells and so forth using apoptosis from transplant tissues used to transplant lenses in cataract treatment (WO96/06863). The polynucleotide of the present invention can be used in such methods as well.

When administered as an antitumor agent, a polynucleotide having a function equivalent to the Msh4 gene splicing variant δ mRNA of the present invention, a fragment thereof, or a vector that encodes the polynucleotide or fragment, demonstrates antitumor effects by inducing or promoting the apoptosis of cancer cells. In addition, it can also be used together with various other types of antitumor agents used in cancer chemotherapy, radiotherapy and other treatment methods as necessary to demonstrate synergistic antitumor effects. Thus, by coordinating administration with other antitumor agents, adequate cancer therapeutic effects can be expected with considerably lower doses of antitumor agents as compared with doses normally used, which can, in turn, reduce side effects associated with antitumor agents that had created problems in the past. Examples of concomitant chemotherapeutic agents include 5-fluorouracil (5-FU), mitomycin, futraful, endoxan and toyomycin.

On the other hand, it is thought that apoptosis may be suppressed by inserting into cells a polynucleotide having antisense effects on the Msh4 gene splicing variant δ mRNA. Here, a “polynucleotide having antisense effects” is only required to suppress the apoptosis inducing effect of the Msh4 gene splicing variant δ mRNA, and is not required to be completely complementary to the nucleotide sequence of the mRNA. Examples of such a polynucleotide having antisense effects include sequences that are complementary to at least about 15 to 20 consecutive residues of the nucleotide sequence set forth in SEQ ID NO: 14.

A region comprising a nucleotide sequence complementary to Bip/Grp78 gene is particularly important as the antisense target sequence of a polynucleotide of the present invention. As indicated in the Examples, apoptosis inducing activity of the Msh4 gene splicing variant δ is either decreased or lost due to deletion or inversion in this region. Thus, an antisense polynucleotide targeting this region is useful in suppression of the apoptosis inducing activity of the Msh4 gene splicing variant δ. Such antisense polynucleotides can be used to treat diseases caused by induction of apoptosis by suppressing the expression of Msh4 gene splicing variant δ in cells. For example, such antisense polynucleotides can be applied to Alzheimer's disease and Alzheimer's senile dementia, etc.

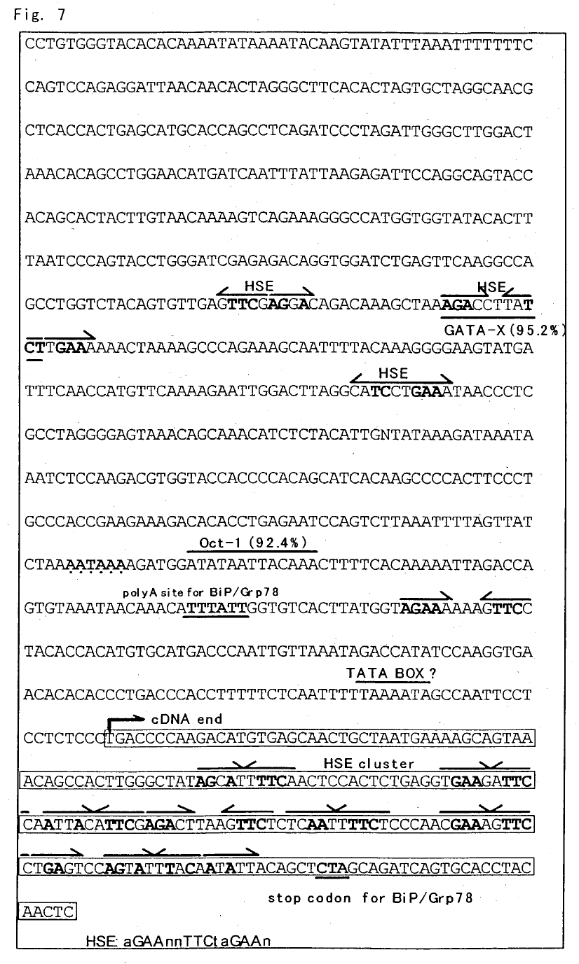

In addition, it may also be possible to control the expression of the Msh4 gene splicing variant δ mRNA in cells by modifying its promoter region. For example, sequencing of the promoter region of the Msh4 gene splicing variant δ (SEQ ID NO: 16) showed that a sequence that is extremely similar to the Heat Shock response Element (HSE) sequence of human HSP70 gene (FIG. 7) which exists in this region. It may also be possible to regulate apoptosis in cells, including nerve cells, by modifying this HSE sequence portion. In addition, apoptosis can also be suppressed by suppressing the expression of the Msh4 gene splicing variant δ mRNA, even when using a factor that inhibits the process of signal transduction upstream from heat shock transcription factor (HSF) which binds to HSE.

The polynucleotide of the present invention or a vector that comprises it may be administered parenterally (intravenous injection, intramuscular injection, drip infusion, etc.) in dosage units containing a known, non-toxic, inert and pharmaceutically acceptable carrier, adjuvant or vehicle. An injection preparation (sterile aqueous solution for injection, oily suspension, etc.) can be prepared using suitable dispersants, lubricants, emulsifiers, pH buffers, preservatives, precipitation-preventing agents, etc. For example, an injection preparation may be produced using a sterile injection solution or suspension of a non-toxic, parenterally acceptable diluent or solvent such as the 1,3-butanediol solution.

Examples of pharmaceutically acceptable vehicles and solvents include water, physiological saline, phosphate buffered saline, Ringer's solution, phosphoric acid and amino acids, polymers, polyoles, sugars, proteins and NaCl isotonic solutions. Moreover, non-irritating sterile oils such as synthetic mono or diglycerides are routinely used as solvents or suspending media. Moreover, fatty acids such as oleic acid may also be used in injection preparations.

Although the required amount of a polynucleotide of the present invention that is used as an antitumor agent can be suitably determined according to the administration method, the patient's age, sex, body weight and the degree of symptoms, and so on, the dose for an adult (with a body weight of 60 kg) is normally about 0.1 to about 500 mg per day when administered daily in single or multiple administrations.

By the present invention, the Msh4 gene splicing variant δ mRNA has been clearly demonstrated to induce apoptosis. Thus, a compound that regulates the action of the Msh4 gene splicing variant δ or a polynucleotide functionally equivalent thereto promotes or suppresses apoptosis. On the basis of such findings, the present invention provides a method for detecting the activity of a test compound that promotes or suppresses apoptosis, comprising:

(i) constructing a cell that expresses the splicing variant δ mRNA of Msh4 gene,

(ii) contacting the cell constructed in (i) with the test compound,

(iii) measuring the apoptosis level and comparing it to a control, and,

(iv) detecting an apoptosis-promoting activity of the compound when the apoptos is level measured in step (iii) has increased in comparison to the control, and detecting an apoptosis-suppressing activity when the apoptosis level measured in step (iii) has decreased in comparison to the control.

In the detection method of the present invention, the, cell of step (i) may be any cell, including the previously mentioned mammalian cells, insect cells, plant cells, yeast and bacterial cells, provided apoptosis is induced by the expression of the Msh4 gene splicing variant δ mRNA of the present invention. Expression of Msh4 gene splicing variant δ mRNA can be achieved by using any of the previously mentioned vectors suitable for expression in the selected cell.

The level of apoptosis can be determined using fluorescence of, for example, EGFP described in Example 4 of the present invention. In addition, methods for measuring apoptosis by trypan blue exclusion assay and by observing changes in cell and nucleus morphology, LDH release, DNA fragmentation (e.g., using in situ end ligation (ISEL) technology), and such are also known.

In the detection method of the present invention, a compound having an apparent effect on the apoptosis-inducing action of splicing variant δ mRNA is used as the control. More specifically, a compound that has been confirmed not to have an effect on the apoptosis-inducing action is used as the control. Alternatively, a compound which clearly has a constant effect may be used as the control, and the magnitude of that effect can be evaluated to see whether it is larger or smaller than the compound.

Moreover, the present invention provides a screening method for compounds having an activity to promote or suppress apoptosis based on the above method for detecting activity of a test compound that promotes or suppresses apoptosis. Namely, the present invention relates to a method of screening for a compound having an activity to promote or suppress apoptosis, comprising:

(i) detecting the activity of a test compound that promotes or suppresses apoptosis by the method for detecting activity of a test compound that promotes or suppresses apoptosis that comprises the above steps (i) through (iii); and,

(ii) selecting a compound that promotes or suppresses apoptosis.

There are no particular restrictions on the test compound that promotes or suppresses apoptosis induced by the Msh4 gene splicing variant δ mRNA. For example, cell extracts, cell culture supernatants, fermenting microorganism products, marine organism extracts, plant extracts, prokaryotic cell extracts, eukaryotic single cell extracts or animal cell extracts, their libraries, purified or crude proteins, peptides, non-peptide compounds, synthetic low molecular weight compounds or naturally-occurring compounds can be used. In the case of using a protein as the test compound of the present invention, the protein can be used as a soluble protein or in a form bound to a carrier. Optionally, it can also be fused with another protein or expressed on a cell membrane depending on the case. In the screening method of the present invention, this type of test compound can be contacted with cells by, for example, adding to a medium. In addition, when the test compound is a nucleic acid, it may also be contacted by injecting into cells.

When apoptosis has significantly decreased in the presence of a test compound, the test compound can be determined to have an activity that suppresses the apoptosis caused by the Msh4 gene splicing variant δ mRNA. A compound that has been selected in this manner is a candidate for a therapeutic or preventive agent for diseases related to apoptosis. Namely, the present invention relates to an apoptosis regulator that contains as its active ingredient a compound selected according to the screening method of the present invention. The apoptosis regulator of the present invention is useful for enhancing and/or regulating the action of the previously indicated pharmaceutical composition containing a polynucleotide of the present invention.

In addition, the present invention provides a nucleotide probe for detecting the Msh4 gene splicing variant δ mRNA as well as a kit containing the probe. Such a probe and kit allow the detection of the Msh4 gene splicing variant δ mRNA in cells, and is able to evaluate whether or not there is a possibility that the cells would undergo apoptosis.

The present invention provides polynucleotides comprising the nucleotide sequence of the Msh4 gene splicing variant δ that induces apoptosis (cell death). Means for regulating the apoptosis in cells using the sequence is also provided, enabling the effective treatment of diseases related to apoptosis.

There is no restriction on length of the nucleic acid of the present invention, but it preferably comprises at least 15, 20, 30, 40, 50, 100, 150, 200, 300, 400, 500, 1000, 1500, 2000, or 2100.

Any patents, patent applications, and publications cited herein are incorporated by reference.

The present invention will be described below with reference to examples, but it is not to be construed as being limited thereto.

### EXAMPLE 1

### Identification of Msh4 cDNA in Mouse Testis

In order to obtain a new member of the MutS family, testis poly(A) RNA was first purified using the FastTrak kit (Invitrogen) from C57BL/6J adult mice (8 weeks old). Reverse transcription PCR (RT-PCR) was carried out on the testis poly(A) RNA using degenerate oligonucleotides based on the amino acid sequences surrounding the ATP-binding domains I and II conserved among MutS family members.

| | | |
|---|---|---|
| I: | 5′-ATIATIACIGGICCIAA(T/C)ATGGG(A/T/G/C)GG(A/T/G/C)AA(A/G)-3′ | (SEQ ID NO: 1) |
| II: | 5′-GCI(G/A)T(T/C)TCIAGCAT(T/C)TCI(G/A)CCAT(A/G)AA-3′ | (SEQ ID NO: 2) |

cDNA was synthesized using a random primer according to the cDNA Cycle Kit (Invitrogen). PCR was carried out for 40 cycles at an annealing temperature of 40-42° C. An approximately 200 bp DNA fragment was amplified by RT-PCR. The resulting cDNA fragment was sub-cloned to pCR2.1 using the TA Cloning Kit (Invitrogen). The cDNA sequence was determined with the ABI 373A Auto DNA Sequencer (Applied Biosystems) by ABI PRISM Dye Terminator Cycle Sequencing.

The amino acid sequence deduced from the cDNA sequence showed homology with the yeast Msh4 (Ross-Macdonald and Roeder, Cell 79: 1069-80 (1994)). Using this cDNA fragment as a probe, mouse testis cDNA library (provided by T. Baba) was screened and cDNA clones around 2 kb, 1.5 kb, and 1 kb were obtained. The amino acid sequences deduced from the cDNA sequences showed significant homology to the yeast Msh4 (Ross-Macdonald and Roeder, Cell 79: 1069-80 (1994)) and the human MSH4 (Paquis-Flucklinger et al., Genomics 44: 188-94 (1997)) but the size of the ORFs, which were 422, 127 and 127 amino acids respectively, were too short for Msh4 which consists of over 800 amino acids. Therefore, the full length cDNA was isolated from testis poly(A) RNA by the Rapid Amplification of cDNA End (RACE) technique using the following nested PCR primers specific for each cDNA.

| | |
|---|---|
| NP-1#30: | |
| 5′-CCATGAGCGAGTGAGTCCAAGACA-3′ | (SEQ ID NO: 3) |
| and | |
| NP-1#31: | |
| 5′-GATGACTTCTGGATGGGGCCATGG-3′ | (SEQ ID NO: 4) |

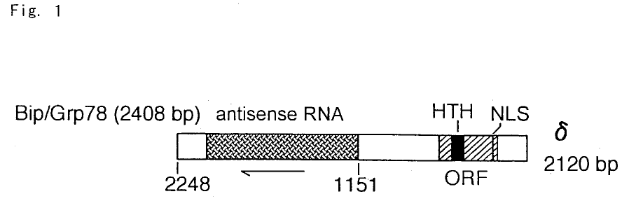

5′RACE-PCR was carried out using the Marathon cDNA Amplification Kit (Clontech). Testis poly(A) RNA was prepared using the FastTrak Kit (Invitrogen) from C57B6L/6J adult mice (8 weeks old), and cDNA was synthesized using an oligo dT primer. 5′RACE-PCR was carried out several times following the usual method described in manuals for obtaining cDNA. The amplified cDNA fragment was cloned to pCR2.1 (Invitrogen), and cDNA sequencing was carried out in the same manner as previously described. The variant δ had antisense RNA of ER chaperon gene Bip/Grp78 (Munro and Pelham, Cell 46: 291-300 (1986)) in its 5′ half, and contained a short ORF (1541-1924) of 127 amino acids (SEQ ID NO: 15) that is commonly observed in the C-terminal region of all Msh4 variants (FIG. 1).

### EXAMPLE 2

### Formation of 6 mRNA by Reverse Splicing and Interchromosomal Trans-Splicing

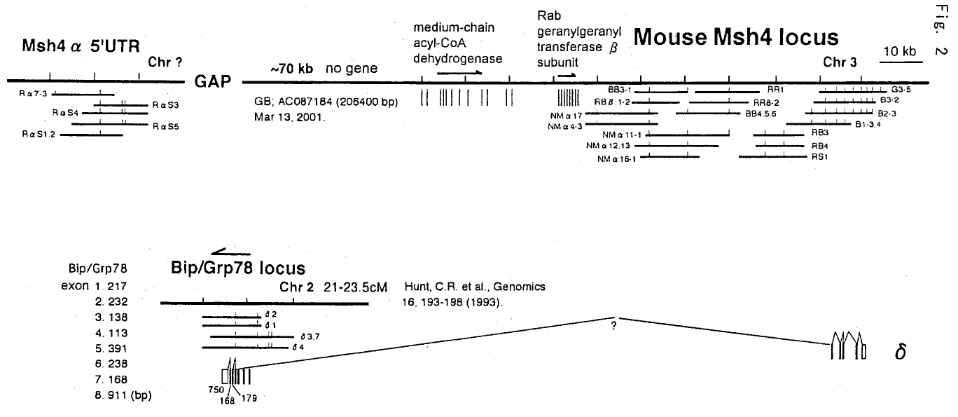

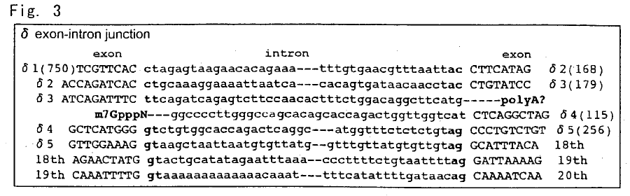

Mouse 129/SvJ genomic library (λFixII vector: Stratagene) was screened using the 5′ half cDNA of α-³²P-labeled variant δ as a probe to isolate genomic DNA clones containing the Bip/Grp78 gene. The NotI genomic DNA fragments purified from the isolated λ phage clones were sub-cloned to pBluescript IIKS(−) (Stratagene), and were subsequently mapped with restriction endonucleases in combination with Southern hybridization using various cDNA fragments as probes. The small DNA fragments containing exon(s) were sub-cloned and the sequences were determined according to the same method as Example 1. The exon-intron structures are shown in FIGS. 2 and 3.

As a result, in the genomic DNA clone corresponding to variant δ, introns were located in exactly the same position as the human BIP/GRP78 gene (Ting and Lee, DNA 7: 275-86 (1988)). The mouse ER chaperon Bip/Grp78 gene, which is one of the five hsp70 family members, has already been mapped to 21-23.5 cM of chromosome 2 (Hunt et al., Genomics 16: 193-8 (1993)). Because the sequence of about 1.1 kb of variant δ cDNA is perfectly complementary to the Bip/Grp78 mRNA sequence determined already (Haas and Meo, Proc. Natl. Acad. Sci. USA 85: 2250-4 (1988)) and the first two introns of variant δ are complementary to the last two introns of Bip/Grp78, the spliceosome must recognize the reverse splice donor and acceptor site (ct-ac) of precursor RNA and exactly splice out these introns (reverse splicing). The next splicing should be carried out in trans between the donor site of Bip/Grp78 antisense RNA precursor and the acceptor site of Msh4 RNA precursor, and then mature chimeric RNA will be completed by following ordinary splicing of four introns. The donor and acceptor site (tt-at) of this interchromosomal trans-splicing is also different from any other donor-acceptor sequences identified so far.

### EXAMPLE 3

### Tissue-Specific Expression of Variant δmRNA

Poly(A) RNA was prepared using the FastTrack Kit (Invitrogen) from the testis, ovary, brain, heart, kidney, thymus, liver and spleen of C57BL/6J adult mice (8 weeks old). 2 μg of poly(A) RNA were separated on a 1.0% agarose formaldehyde gel and transferred to a Nylon membrane. α-³²P dCTP-labeled mouse Msh4γ cDNA, which hybridizes to all Msh4 variants, and chicken β-actin cDNA, were used as probes for hybridization.

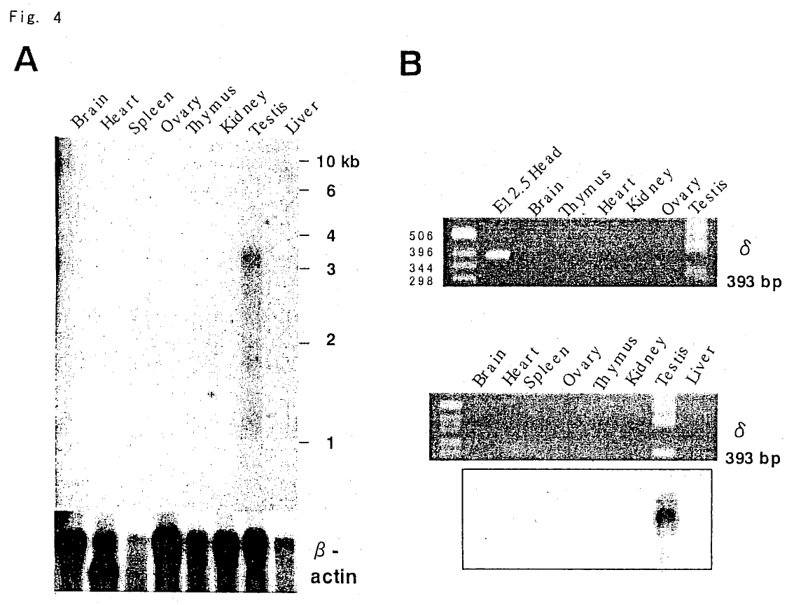

As a result, Msh4 expression was shown to be limited to the testis (FIG. 4A). In order to investigate lower levels of variant-specific expression in other tissues at higher sensitivity, RT-PCR was carried out using variant-specific oligonucleotide as the primer. Poly(A) RNA were prepared from testis, ovary, brain, heart, kidney, thymus, liver and spleen of C57BL/6J adult mouse (8 week old) and E12.5 embryonic head using FastTrack kit (Invitrogen). First strand cDNA were synthesized by cDNA cycle kit using a random primer (Invitrogen). Primers used for RT-PCR were listed below:

| | | | |
|---|---|---|---|
| δ specific; | #17 | 5′-CCAGGTCACATGGCATCAGACTAG-3′ | (SEQ ID NO: 5) |
| and | | | |
| | #18 | 5′-GGCATAGTTCCAGGAAATGGGTAG-3′ | (SEQ ID NO: 6) |
| β-actin; | β-actin 1: | 5′-GTGACGAGGCCCAGAGCAAGAG-3′ | (SEQ ID NO: 7) |
| and | | | |
| | β-actin 2: | 5′-AGGGGCCGGACTCATCGTACTC-3′. | (SEQ ID NO: 8) |

PCR was carried out for 35 or 40 cycles at appropriate annealing temperatures, 63 to 70° C.

As a result, variant δ was shown to be expressed in the testis and E12.5 embryonic head (FIG. 4B). Msh4 gene is well known to be involved in the meiotic recombination process, the products of which are localized to the zygotene chromosome (Kneitz et al., Genes Dev. 14: 1085-97 (2000)) and synaptonemal complex of the pachytene chromosome (Ross-Macdonald and Roeder, Cell 79: 1069-80 (1994)). Msh4 variant δ protein obtained along with variant δ is likely to be involved in the meiotic recombination process. However, the function of variant δ expressed at lower levels is unknown. Therefore, the inventors investigated whether the presumably small ORF of variant δ is translated in vivo.

### EXAMPLE 4

### Constitutive Expression of Variant δ mRNA

To test the physiological role of the variant δ mRNA containing the Bip/Grp78 antisense RNA and small ORF in cells, the inventors constructed an expression vector for producing the Bip/Grp78 antisense RNA and small ORF-EGFP fusion protein. First, the full length cDNA was constructed according to the method of Example 1, and then, the full length cDNA was inserted into pEGFP—N₁ vector (Clontech) by recombinant DNA technologies using restriction endonucleases and enzymes for modifying nucleic acids (Sambrook et al., “Molecular Cloning: A Laboratory Maunal, 2^{nd} ed.” Cold Spring Harbor, N.Y., Cold Spring Harbor Laboratory Press (1989)) to construct the EGFP fusion gene.

The junction sequences of the EGFP fusion gene are shown below.

| | | | |
|---|---|---|---|
| BbsI | | NcoI | |
| Msh4δ --GAG AAG ACT GAG GAG TGA---, | (SEQ ID: 9) | EGFP-N1--CC ATG G-- | (SEQ ID: 10) |
| E K T E E | (SEQ ID: 11) | | |
| C53CE1 GAG AAG ACT GAG GAC ATG | (SEQ ID: 12) | fusion at C-terminal of δORF | |
| E K T E D M | (SEQ ID: 13) | | |

Plasmids C53-1CE1 and C53-2CE1 were constructed by deletion and inversion of a 1251 bp BamHI-BglII fragment of C53CE1 containing the Bip/Grp78 antisense RNA region. The CAGS-nlacZ plasmid used as the internal standard has lacZ gene that carries the nuclear localization signal of SV40 T antigen driven by the chicken β-actin promoter.

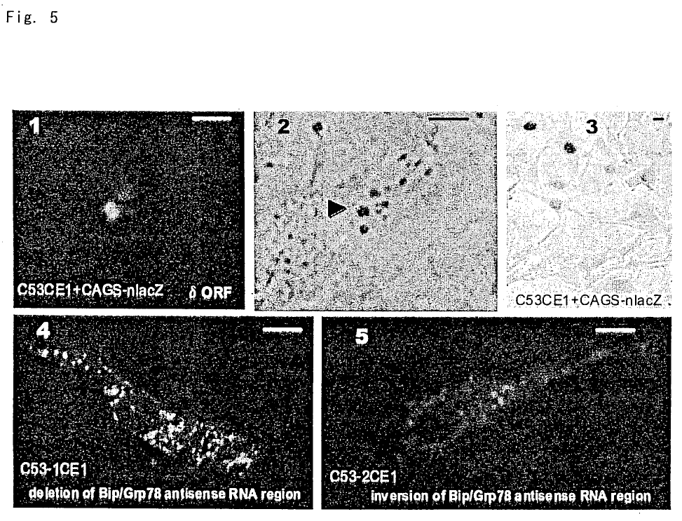

First, 1 to 2×10⁵ NIH3T3 cells were grown in a six-well tissue culture plate containing 2 ml of growth medium (DMEM: Sigma) supplemented with serum (10% FCS: Gibco BRL). When the cells had become 40-60% confluent, co-transfection was carried out with the 6-EGFP fusion plasmid and CAGS-nlacZ plasmid by a lipofectin reagent (Life Technologies)-DNA (1 μg) complex according to the method described in the instruction manual. The cells were then observed with a fluorescent microscope (Olympus IX70) two days after transfection. As a result, only small cell populations expressed the fusion protein, but many cells expressed β-galactosidase (β-gal) (FIGS. 5-1 to 5-3). Deletion and inversion of the Bip/Grp78 antisense RNA region of variant δ cDNA caused an increase in the cell number expressing EGFP fusion protein, and the expressed proteins were localized to a certain cytoplasmic compartment (FIGS. 5-4 and 5-5). A more close observation showed that in the deletion and inversion constructs, EGFP expressing cells formed patches consisting of several cells by two to three cell divisions, while cells producing Bip/Grp78 antisense RNA did not form distinct patches, but only one to few cells emitted fluorescence.

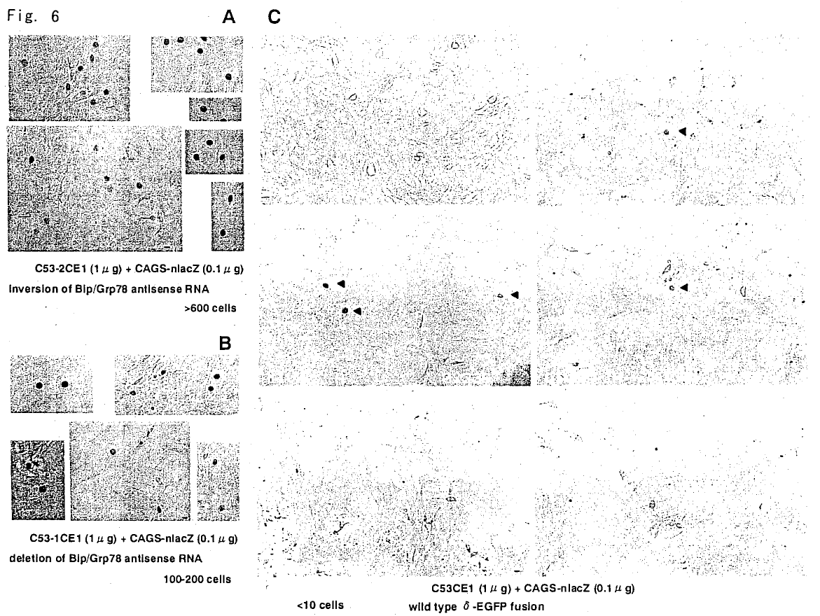

Moreover, co-transfection was carried out after reducing the 6-EGFP fusion plasmid:CAGS-nlacZ plasmid ratio to 0.1 (1 μg and 0.1 μg, respectively). The cells were fixed and stained with X-Gal to investigate the expression of β-galactosidase. The number of cells showing both β-gal and 6-mRNA expression was dramatically diminished to about 1/100 level seen as a result of the co-transfection of the inversion mutant of the Bip/Grp78 antisense RNA region of 6-EGFP and CAGS-nlacZ plasmids with the same ratio (FIG. 6). These observations indicate that the coexistence of Bip/Grp78 antisense RNA and β-gal producing plasmids in the same cell causes a reduction of X-Gal stained cells. Taken together, the constitutive expression of the variant δ mRNA seems likely to induce cell death. When using a plasmid containing a mutant in which the antisense RNA region in FIG. 6B had been deleted, the number of β-Gal expressing cells decreased to ¼^{th} to ⅕^{th} of the level when using a plasmid containing an inversion mutant. The reason behind this is presumably an increased translation of the ORF coding for the 127 amino acid residues resulting from a deletion of the antisense region.

### INDUSTRIAL APPLICABILITY

The present invention discloses polynucleotides that induce apoptosis (cell death) by forming a double strand with the ER chaperon Bip/Grp78 mRNA, particularly the Msh4 gene splicing variant δ and functional equivalents thereof. Such polynucleotides, as well as DNA coding for the same, fragments thereof, and expression vectors containing the DNA, find utility in pharmaceutical compositions as gene therapy agents, temporarily inducing apoptosis to select tissues, such as cancer tissues. The polynucleotide of the present invention or a vector that expresses the polynucleotide is preferably applied directly to the desired apoptosis site. However, it may also be made to ultimately arrive at the desired affected tissue following intravascular administration.

Since it has the ability to induce apoptosis, a polynucleotide or a fragment thereof having a function that is equivalent to the Msh4 gene splicing variant δ mRNA of the present invention can be applied to various diseases, such as cancer, AIDS, and autoimmune diseases. The pharmaceutical composition of the present invention can also be used in the treatment of diseases that accompany decreased platelet levels, in which by inhibiting the proliferation of blast cells, the compound would be able to promote the proliferation of mature cells. The pharmaceutical composition of the present invention may be administered alone or in combination with other known therapies, to achieve a synergistic effect.

Antisense molecules that suppress the expression of the Msh4 gene splicing variant δ or its apoptosis inducing activity can be used to treat diseases caused by induction of apoptosis. For example, such antisense polynucleotides can be applied to Alzheimer's disease and Alzheimer's senile dementia, etc.

What is claimed:

1. An isolated polynucleotide that forms a double-stranded moiety with an mRNA encoding ER chaperon Bip/Grp78, said polynucleotide selected from the group consisting of:
(i) the nucleotide sequence set forth in SEQ ID NO:14;
(ii) the nucleotide sequence of (i), wherein one or more residues have been deleted, inserted, substituted and/or added to a 5′ half of said sequence, the 5′ half comprising nucleotide 1-1098 in the nucleotide sequence set forth in SEQ ID NO:14;
(iii) a nucleotide sequence complementary the nucleotide sequence of (i) that hybridizes thereto under stringent conditions, wherein said stringent hybridization conditions comprise: prehybridizing for at least 30 minutes at 68° C., adding a labeled probe and hybridizing for at least 1 hour at 68° C., followed by washing three times for 20 minutes at room temperature in 2×SSC, 0.01% SDS, then three times for 20 minutes at 37° C. in 1×SSC, 0.1% SDS, and finally twice for 20 minutes at 50° C. in 1×SSC, 0.1% SDS;
(iv) a nucleotide sequence having 90% or more homology with a 5′ half of the nucleotide sequence of (i) and 50% or more homology with a 3′ half of the nucleotide sequence of (i), wherein the line between the 5′ and 3′ halves of the sequence arises at nucleotide 1098 of SEQ ID NO:14; and
(v) a polynucleotide having a nucleotide sequence comprising at least 25 consecutive nucleotides selected from nucleotides 1-1098 in the nucleotide sequence set forth in SEQ ID NO: 14, wherein said fragment is perfectly complementary to a nucleotide sequence corresponding to the mRNA encoding Bip/Grp78.

2. A DNA encoding the splicing variant δ mRNA of Msh4 gene.

3. An expression vector comprising the DNA of claim 2.

4. The expression vector of claim 3, wherein the DNA of claim 2 is linked downstream of a tumor-specific promoter and/or a tumor-specific transcription regulatory sequence.

5. A pharmaceutical composition comprising an active agent selected from the group consisting of the polynucleotide of claim 1, the DNA of claim 2, the expression vector of claim 3, and the expression vector of claim 4.

6. The pharmaceutical composition of claim 5, wherein said active agent is present in an amount effective to induce or promote apoptosis in cancer cells.

7. A method for regulating apoptosis in a target cell, wherein the method comprises the step of introducing into the target cell a construct comprising a polynucleotide selected from the group consisting of:
(i) the nucleotide sequence set forth in SEQ ID NO: 14;
(ii) the nucleotide sequence of (i), wherein one or more residues have been deleted, inserted, substituted and/or added to a 5′ half of said sequence, the 5′ half comprising nucleotide 1-1098 in the nucleotide sequence set forth in SEQ ID NO:14;
(iii) a nucleotide sequence complementary the nucleotide sequence of (i) that hybridizes thereto under stringent conditions, wherein said stringent hybridization conditions comprise: prehybridizing for at least 30 minutes at 68° C., adding a labeled probe and hybridizing for at least 1 hour at 68° C., followed by washing three times for 20 minutes at room temperature in 2×SSC, 0.01% SDS, then three times for 20 minutes at 37° C. in 1×SSC, 0.1% SDS, and finally twice for 20 minutes at 50° C. in 1×SSC, 0.1% SDS;
(iv) a nucleotide sequence having 95% or more homology with a 5′ half of the nucleotide sequence of (i) and 50% or more homology with a 3′ half of the nucleotide sequence of (i), wherein the line between the 5′ and 3′ halves of the sequence arises at residue 1098 of SEQ ID NO:14; and
(v) a polynucleotide having a nucleotide sequence comprising at least 25 consecutive nucleotides selected from nucleotides 1-1098 in the nucleotide sequence set forth in SEQ ID NO: 14, wherein said fragment is perfectly complementary to a nucleotide sequence corresponding to the mRNA encoding Bip/Grp78.

8. A method for detecting the activity of a test compound to promote or suppress apoptosis, wherein the method comprises the following steps of:
(i) constructing a cell that expresses the splicing variant δ mRNA of Msh4 gene,
(ii) contacting the cell constructed in (i) with a test compound,
(iii) measuring the level of apoptosis in the cells and comparing it to a control, and,
(iv) detecting an apoptosis-promoting activity of the compound when the apoptosis level measured in step (iii) is increased as compared to the control, and detecting an apoptosis-suppressing activity when the apoptosis level measured in step (iii) is decreased as compared to the control.

9. A method of screening for a compound having an activity to promote or suppress apoptosis, comprising:
(i) detecting the apoptosis activity of a test compound by the method of claim 8, and
(ii) selecting the compound that promotes or suppresses apoptosis.

10. A nucleotide probe comprising at least 25 consecutive nucleotides from the nucleotide sequence set forth in SEQ ID NO: 14, wherein said probe hybridizes under stringent conditions to the splicing variant δ mRNA of Msh4 gene, said stringent hybridization conditions comprising: prehybridizing for at least 30 minutes at 68° C., adding a labeled probe and hybridizing for at least 1 hour at 68° C., followed by washing three times for 20 minutes at room temperature in 2×SSC, 0.01% SDS, then three times for 20 minutes at 37° C. in 1×SSC, 0.1% SDS, and finally twice for 20 minutes at 50° C. in 1×SSC, 0.1% SDS.

11. A kit for detecting apoptosis in cells by detecting the presence of the splicing variant δ mRNA of Msh4 gene comprising the nucleotide probe of claim 10.

12. A method for detecting apoptosis in cells comprising the step of detecting the presence of the splicing variant δ mRNA of Msh4 gene by the nucleotide probe of claim 10.

13. A polynucleotide that (a) comprises at least 25 consecutive nucleotides from the nucleotide sequence set forth in SEQ ID NO: 16, and (b) regulates the transcription of the splicing variant δ of Msh4 gene.
